(19) United States
(12) Patent Application Publication
(10) Pub. No.: US 2011/0086797 A1
(43) Pub. Date: Apr. 14, 2011

(54) CONTROL OF SPORE GERMINATION

(75) Inventor: Jonathan Dworkin, New York, NY (US)

(73) Assignee: The Trustees of Columbia University in the City of New York, New York, NY (US)

(21) Appl. No.: 12/748,148

(22) Filed: Mar. 26, 2010

Related U.S. Application Data
(63) Continuation-in-part of application No. PCT/US08/78004, filed on Sep. 26, 2008.
(60) Provisional application No. 60/975,399, filed on Sep. 26, 2007.
(60) Provisional application No. 61/060,773, filed on Jun. 11, 2008.
(60) Provisional application No. 61/075,273, filed on Jun. 24, 2008.

(51) Int. Cl. A61K 38/02; A61P 31/04; A01N 37/18; C12N 1/20; A61K 31/65; A61K 31/366; A61K 31/553; A01N 43/72; A01N 43/16; C12N 1/38

(52) U.S. Cl. 514/3.1; 435/252.5; 514/154; 514/453; 514/211.08; 435/244

(57) ABSTRACT

Provided are compositions and methods for treating bacterial infections. It is demonstrated herein that bacteria cell wall materials stimulate germination of spores of Gram-positive bacteria, and that such activity requires Ser/Thr kinase PrkC. By modulating one or both, spores (which can be antibiotic resistant) can be stimulated or inhibited from germination, which can be exploited in various methods of therapeutic treatment. Also provided is a method of modulating germination of a spore of a Gram-positive bacterium. Also provided is a method of decontaminating an environment.

### CROSS-REFERENCE TO RELATED APPLICATIONS

This application is a Continuation-in-Part of International Application No. PCT/US08/78004, filed on Sep. 26, 2008, which in turn claims the benefit of U.S. Provisional Application No. 60/975,399, filed Sep. 26, 2007, U.S. Provisional Application No. 61/060,773, filed Jun. 11, 2008, and U.S. Provisional Application No. 61/075,273, filed Jun. 24, 2008, each of which is incorporated herein by reference in its entirety.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to spore-forming Gram-positive bacteria.

### BACKGROUND

Peptidoglycan (PG) fragments and resuscitation of dormant bacterial cells. Micrococcus luteus cells in a prolonged stationary phase culture enter a dormant state (Kaprelyants and Kell, 1993). These dormant cells can be stimulated to divide (resuscitate) by exposure to non-dormant M. luteus cells (Votyakova et al., 1994). Resuscitation requires the resuscitation-promoting factor (Rpf), a secreted 17-kDa protein that has growth promoting actions at low-picomolar concentrations (Mukamolova et al., 2002, 2006). The predicted structure of the conserved domain of Rpf as similar to lysozyme (Cohen-Gonsaud et al., 2004) has been confirmed by the NMR structure of M. tuberculosis RpfB (Cohen-Gonsaud et al., 2005). Rpf from M. luteus is a muralytic enzyme that causes lysis of E. coli when expressed and secreted in the periplasm. Thus, the biological activity of Rpf is thought to result directly or indirectly from its ability to cleave bonds in bacterial PG (Mukamolova et al. 2006).

In silico analysis of the accessory domains of Rpf proteins classifies them into several subfamilies and the RpfB subfamily is related to a group of firmicute proteins of unknown function in B. subtilis by YabE, YocH, and YuiC (Ragavani et al., 2005). It is uncertain the nature of the signal that Rpf generates, how the signal is relayed to or detected by a cell, and the steps in the pathway from Rpf action at the cell surface to the relief of growth arrest (Keep et al., 2006).

Biological function of PG derived muropeptides. Molecular mechanisms(s) of immunostimulatory activities of PG and its muropeptide derivatives are not fully understood. Muropeptides resulting from PG cleavage have been recognized as critical factors in host recognition of bacterial pathogens. For example, in Drosophila, where the PGRP (Peptidoglycan receptor proteins) are involved in the activation of immune responses, bacterial cell wall PG molecules containing the diaminopimelic acid (DAP) sugar from Gram-negative bacteria and Bacilli are sensed during the process of bacterial infection. This mechanism of pattern recognition allows flies to distinguish between the DAP-containing PG and lysine-containing PG from Gram-positive bacteria (Filipe et al., 2005).

Bacterial cell wall recycling. Gram-negative bacteria recycle their cell wall PG by reutilizing PG degradation products resulting from the action of hydrolases. E. coli degrades half of its PG layer during exponential growth, releasing ˜5% of the material in the environment (Park, 1995). In E. coli, specific permeases transport muropeptides resulting from degradation of cell wall PG and they are induced by some antibiotics that disrupt PG synthesis (Jacobs et al., 1997). For Gram positive bacteria, ˜50% of their cell wall material is released into the extracellular milieu. PG turnover caused by the action of PG hydrolases results in shedding of the cell wall in the environment (Boneca, 2005).

Cell-cell signaling. Bacteria can control their behavior in response to cell number variations by producing, releasing, exchanging and detecting signaling molecules to measure population density (Bassler and Losick, 2006). Examples include chemically modified short-peptides like the genetic competence factor ComX of B. subtilis, a 6 amino acid peptide. ComX is recognized by the membrane bound two-component sensor kinases ComP and the resulting signal is transduced via a phosphorylation cascade (Bassler and Losick, 2006). Tracheal cytotoxin (TCT), a fragment of PG from V. fischeri is capable of inducing normal light organ morphogenesis in the squid host, demonstrating that bacteria can signal eukaryotic hosts via the release of PG (Koropatnick et al., 2004).

The bacterium Mycobacterium tuberculosis (Mtb) is the cause of the most prevalent bacterial infection in the world, with an estimate of >1 billion infected individuals.

Mtb kinase has been considered a drug target (Fernandez et al., 2006) but it is insensitive in vivo to some commercially available kinase inhibitors. There are also numerous difficulties in using either in vitro or in vivo strategies to identify compounds that target Mtb kinase. In vitro assays are limited by their inability to assay permeability of compounds into the bacterial cell. And Mtb can be difficult to work with in vivo, given its replication time of about 8 hours.

To date, there are no known compounds that can inhibit the ability of Mtb to reactivate.

### SUMMARY OF THE INVENTION

One aspect provides a method of treating an infection of a spore-forming Gram-positive bacterium.

In various embodiments, the method of treatment includes administering an effective amount of a first composition for stimulating germination of spore of a first Gram-negative bacterium to a subject in need thereof. In some embodiments, the first composition comprises at least one of (i) a peptidoglycan fragment or muropeptide of a second bacterium or a preparation comprising cell walls from the second bacterium; (ii) a compound that stimulates activity of a bacterial serine/threonine protein kinase; or (iii) a compound that inhibits activity of a bacterial PPM-like phosphatase; wherein the first composition stimulates germination of the spore.

In various embodiments, the method of treatment includes administering an effective amount of a second composition for inhibiting germination of the spore to a subject in need thereof. In some embodiments, the a second composition comprises at least one of (iv) a compound that inhibits activity of a bacterial serine/threonine protein kinase; or (v) a compound that stimulates activity of a PPM-like phosphatase of a first Gram-positive bacterium; wherein the second composition inhibits germination of the spore.

In various embodiments, the method of treatment includes administering an antibiotic to the subject. In some embodiments, the antibiotic is effective against a Gram-positive bacterium, such as the first Gram-positive bacterium.

In various embodiments, a composition including the antibiotic and at least one of (i), (ii), or (iii), as described above, is administered to the subject. In some embodiments, the composition of an antibiotic and (i), (ii), or (iii) is a pharmaceutical formulation. In some embodiments, the composition of an antibiotic and (i), (ii), or (iii) also includes a pharmaceutically acceptable carrier or excipient.

In various embodiments, the subject is a mammal. In some embodiments, the subject is a mouse, rat, guinea pig, gerbil, dog, cat, sheep; cow; horse; pig; goat; donkey; mule; monkey; prosimian; ape; or human.

Another aspect provides a pharmaceutical composition.

In various embodiments, the pharmaceutical composition includes an antibiotic; a first composition including at least one of (i) a peptidoglycan fragment or muropeptide of a second bacterium or a preparation comprising cell walls from the second bacterium; (ii) a compound that stimulates activity of a serine/threonine protein kinase of a Gram-positive bacterium; or (iii) a compound that inhibits activity of a PPM-like phosphatase of a Gram-positive bacterium; wherein the first composition stimulates germination of a Gram-positive bacterium spore; and a pharmaceutically acceptable carrier or excipient. In some embodiments, the antibiotic is effective against a Gram-positive bacterium.

In various embodiments, the pharmaceutical composition includes an antibiotic; a second composition including at least one of (iv) a compound that inhibits activity of a bacterial serine/threonine protein kinase; or (v) a compound that stimulates activity of a PPM-like phosphatase of a first Gram-positive bacterium; wherein said second composition inhibits germination of a Gram-positive bacterium spore; and a pharmaceutically acceptable carrier or excipient.

Another aspect provides a method of decontaminating an environment containing spores of a Gram-positive bacterium. In various embodiments, the decontamination method includes treating the environment with a composition including at least one of (i) a peptidoglycan fragment or muropeptide of a second bacterium or a preparation comprising cell walls from the second bacterium; (ii) a compound that stimulates activity of a serine/threonine protein kinase of a Gram-positive bacterium; or (iii) a compound that inhibits activity of a PPM-like phosphatase of a Gram-positive bacterium.

Another aspect provides a method of modulating germination of a spore of a Gram-positive bacterium.

In various embodiments, the modulation method includes contacting the spore of the first Gram-positive bacterium with a first composition including at least one of (i) a peptidoglycan fragment or muropeptide of a second bacterium or a preparation comprising cell walls from the second bacterium; (ii) a compound that stimulates activity of a serine/threonine protein kinase of a Gram-positive bacterium; or (iii) a compound that inhibits activity of a PPM-like phosphatase of a Gram-positive bacterium; wherein the first composition stimulates germination of the spore.

In various embodiments, the modulation method includes contacting the spore of the first Gram-positive bacterium with a second composition including at least one of (iv) a compound that inhibits activity of a bacterial serine/threonine protein kinase; or (v) a compound that stimulates activity of a PPM-like phosphatase of a first Gram-positive bacterium; wherein the second composition inhibits germination of the spore.

In various embodiments, the first Gram-positive bacterium is Bacillus, Clostridium, Desulfotomaculum, Sporolactobacillus, Sporosarcina, or Thermoactinomyces. In some embodiments, the first Gram-positive bacterium is B. anthracis, B. cereus, B. thuringiensis, C. difficile, C. botulinum, B. subtilis, B. megaterium, B. anthracis, or C. acetobutylicum.

In various embodiments, the second bacterium is a second Gram-positive bacterium. In some embodiments, the second Gram-positive bacterium is Bacillus, Clostridium, Listeria, or Streptomyces. In some embodiments, the second Gram-positive bacterium is B. subtilis, B. megaterium, B. anthracis, C. acetobutylicum, L. monocytogenes, or S. coelicolor. In some embodiments, the first Gram-positive bacterium is of the same genus or the same species as the second Gram-positive bacterium.

In various embodiments, the second bacterium is a second Gram-negative bacterium. In some embodiments, the second Gram-negative bacterium is E. coli.

In various embodiments, the preparation of cell walls includes purified peptidoglycan fragments or purified muropeptides. In some embodiments, the preparation of cell walls includes purified peptidoglycan fragments. In some embodiments, the preparation of cell walls includes muropeptides. In some embodiments, the purified peptidoglycan fragments or purified muropeptides comprise a diaminopimelic acid (DAP) in the third residue of the stem-peptide.

In various embodiments, the preparation of cell walls does not contain a living bacterium.

In various embodiments, the compound that stimulates activity of a bacterial serine/threonine protein kinase is a compound that stimulates activity of PrkC or a polypeptide having an amino acid sequence at least 25% identical to the sequence encoded by a complement of nucleotides 2106-4033 of SEQ ID NO:1.

In various embodiments, the compound that stimulates activity of a bacterial serine/threonine protein kinase is a phorbol ester; phorbol-12-myristate-13-acetate (PMA); a bryostatin; or teleocidin; or a derivative thereof.

In various embodiments, the antibiotic is a beta-lactam, clavulanic acid, a monobactam, a carboxypenem, an aminoglycoside, gentamicin, a glycopeptide, a lincomycin, a macrolide, bacitracin, rifamycin, tetracycline, chloramphenicol, penicillin G, benzathine benzylpenicillin, phenoxymethylpenicillin, amoxicillin, ampicillin, bacampicillin, pivampicillin, clavulanic acid, aztreonam, imipenem, streptomycin, vancomycin, clindamycin, erythromycin, bacitracin, rifampicin, doxycycline, tigecycline, chloramphenicol, linezolid, quinupristin-dalfopristin, or daptomycin.

In various embodiments, the compound that inhibits activity of a bacterial serine/threonine protein kinase (see (iv) of the second composition described above) is adaphostin, AG 490, AG 825, AG 957, AG 1024, aloisine, aloisine A, alsterpaullone, aminogenistein, API-2, apigenin, arctigenin, AY-22989, bisindolylmaleimide IX, chelerythrine, DMPQ, DRB, edelfosine, erbstatin analog, ET18OCH3, ERK inhibitor fasudil, gefitinib, H-7, H-8, H-89, HA-100, HA-1004, HA-1077, HA-1100, Heatstable protein kinase A inhibitor PKI, hydroxyfasudil, indirubin-3′-oxime, 5-lodotubercidin, kenpaullone, KN-62, KY12420, LFM-A13, luteolin, LY-294002, LY294002, mallotoxin, ML-9, NSC-154020, NSC-226080, NSC-231634, NSC-664704, NSC-680410, NU6102, olomoucine, oxindole I, PD 153035, PD 98059, PD 169316, phloridzin, piceatannol, picropodophyllin, PKI, PP1, PP2, purvalanol A, quercetin, RAPA, rapamune, rapamycin, Ro 31-8220, roscovitine, rottlerin, SB202190, SB203580, sirolimus, SL327, SP600125, staurosporine, STI-571, SU1498, SU4312, SU6656, syk inhibitor, TBB, TCN, triciribine, Tyrphostin AG 490, Tyrphostin AG 825, Tyrphostin AG 957, Tyrphostin AG 1024, U0126, W-7, wortmannin, Y-27632, ZD 1839, and ZM 252868.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a graph of experimental results showing the induction or repression of various genes in B. subtilis following exposure to cell wall fragments.

FIG. 2 is a diagram, a graph, and a photograph of a zymogram showing various characteristics of the B. subtilis YocH protein. Panel A shows a comparison of a portion of the amino acid sequences of YocH and other proteins in the MltA family. Panel B is a graph showing the lysis of bacterial cells in the presence of YocH and hen egg white lysozyme (HEWL). Panel C is a zymogram showing clearance at the appropriate molecular weight.

FIG. 3 is a diagram and a graph showing the dependence of the induction of yocH to PrkC after exogenous cell wall exposure. Panel A is a diagram of the PrkC protein. Panel B is a graph showing the induction of yocH.

FIG. 4 is diagrams, a photograph of a blot, and a graph showing that the extracellular domain of PrkC binds cell wall. Panel A is a diagram showing PrkC and another B. subtilis membrane protein, Yycl. Panel B is a diagram depicting the his₆-tagged extracellular domain of PrkC and Yycl. Panel C is a Coomassie-stained gel of showing total purified protein that is added to the cell wall fraction (UN), the protein that comes off when the cell wall fraction is washed (W) and the protein that is bound to the cell wall fraction (B). The B fraction was generated by adding SDS to the cell wall to release bound protein. Panel D is a graph showing that PrkC binds to cell wall much better than Yycl.

FIG. 5 is a diagram showing a model of cell wall binding to PrkC and induction of yocH.

FIG. 6 are electron micrographs (Panel A) and phase contrast micrographs (Panel B) showing germination of Gram positive spores.

FIG. 7 is a graph (Panel A) and micrographs (Panel B) showing the effect of purified cell wall from B. subtilis on spore germination.

FIG. 8 is micrographs (Panel A) and a graph (Panel B) showing the effect of cell wall preparations from various bacteria on spore germination.

FIG. 9 is micrographs (Panel A) and a graph (Panel B) showing the effect of cell wall preparations from various bacteria on germination of B. megaterium and B. anthracis spores.

FIG. 10 is a graph showing that cell wall induced germination does not use the same molecular mechanism as nutrient germination.

FIG. 11 is a graph (Panel A) and micrographs (Panel B) showing that spores derived from a strain lacking PrkC (ΔprkC) do not germinate in response to cell wall, although they still respond to alanine.

FIG. 12 is micrographs (Panel A) and a graph (Panel B) showing that supernatant from growing cells acts to induce germination.

FIG. 13 is a diagram depicting the PrkC signaling pathway.

FIG. 14 is a graph showing that the phorbol ester PMA induces germination.

FIG. 15 is a graph (Panel A) and a ribbon diagram (Panel B) showing the inhibitory effect of staurosporine on spore germination, and a co-crystal structure of staurosporine binding to a protein kinase, showing that staurosporine binds in the ATP pocket.

FIG. 16 is a diagram depicting a model of the stimulation of spore germination by cell walls.

FIG. 17 is a diagram of peptidoglycan structure in various bacterial species. Panel A shows B. subtilis peptidoglycan, which is composed of chains of N-acetylglucosamine (GlcNAc) and N-acetylmuramic acid (MurNAc) attached to stem peptides. Bonds between m-Dpm and D-ala residues arising from separate chains cross-link the GlcNAc-MurNAc polymers. The vast majority of the D-Ala residues that are not in crosslinks (>95%) are removed, leaving the tripeptides, and only 40% of the peptides are cross-linked. Mutanolysin (red) hydrolyzes the β-1,4 bond between the MurNAc and GlcNAc sugars. Panel B. Most Gram-positive bacteria (e.g. S. aureus) contain an L-lys residue at the 3rd position of the stem peptide (left). Gram-negative bacteria and most spore-formers (except B. sphaericus) have an m-Dpm residue in this position (right). Panel C shows the structure of the disaccharide tripeptide.

FIG. 18 is graphs showing that peptidoglycan germinates bacterial spores. Panel A shows germination results from cell free supernatant prepared from growing B. subtilis PY79 (squares), E. coli DH5α (circles) or S. aureus Newman (diamonds) at a range of dilutions incubated with B. subtilis spores for 60 min. Panel B shows germination results from B. subtilis mutanolysin-digested peptidoglycan at a range of concentrations incubated with wild type B. subtilis spores for 60 min. Panel C shows germination results from a disaccharide tripeptide at a range of concentrations incubated with wild type B. subtilis spores for 60 min. Error bars represent s.d. for triplicate samples.

FIG. 19 is phase contrast images of cells exposed to germinants. Wild type PY79 spores (wt), FB85 spores lacking all five nutrient germination receptors (Δger5) or PB705 spores lacking PrkC (ΔprkC) were incubated with germination buffer alone or with 10 mM L-alanine (Alanine), 1 μg/ml B. subtilis peptidogylcan (PG), or B. subtilis cell free supernatant (CFS, 10⁻³ dilution) for 60 min and 100× phase contrast images were subsequently acquired.

FIG. 20 is a graph showing kinetics of germination. Wild type PY79 spores were incubated with germination buffer alone or with germination buffer containing 1 mM L-alanine (▪) or cell free supernatant (♦) for times indicated and the percentage of heat sensitive (80° C., 20 min) spores was determined.

FIG. 21 is a graph showing the effect on percent germination of cell free supernatant isolated from non-growing cells on spore germination. B. subtilis cells were grown up to an A600 of 1.2, washed and transferred to non-growth promoting buffer (Tbase/10 mM MgSO₄) and incubated for 24 hours. Filtrate (CFS(NG)) was subsequently isolated and used in a germination assay as described in Experimental Procedures along with L-alanine (1 mM) and cell-free supernatant (CFS) prepared as described in Experimental Procedures as controls.

FIG. 22 is a graph showing the effect on percent germination of cortex peptidoglycan on spore germination. PG from decoated spores was obtained as described in Experimental Procedures for vegetative PG by boiling in 4% SDS and washing extensively with dH20. The resulting suspension was used at indicated concentrations in a germination assay.

FIG. 23 is a graph showing the effect of a ΔprkC mutation on Ca²⁺-DPA spore germination. Wild type PY79 and PB705 ΔprkC B. subtilis spores were incubated with 1 mM L-alanine, 100 μg/ml PG, or 50 mM Ca²⁺-DPA (Sigma) for 60 min and % germination was determined.

FIG. 24 is a diagram, graphs, and a western blot showing that peptidoglycan-dependent germination uses a novel signal transduction pathway. As Panel A illustrates, PrkC consists of an N-terminal kinase domain, a membrane spanning sequence and three PASTA repeats in the extracellular domain. Panel B shows % germination when wild type or ΔprkC spores is incubated with L-alanine (1 mM), B. subtilis peptidoglycan (100 ng/ml), or B. subtilis disaccharide tripeptide (‘tri’; 10 μM) for 60 min. Panel C shows % germination when wild type or ΔprkC spores are incubated with undiluted cell free supernatant prepared from log-phase B. subtilis (Bs) or E. coli DH5α (Ec) for 60 min. Panel D shows blots when protein lysates from B. subtilis ΔprkC or wild type spores were incubated with buffer alone (−) or with B. subtilis cell free supernatant (CFS; 10⁻³ dilution) for 60 min then immunoprecipitated with α-EF-G antibodies and subjected to western blotting with either α-EF-G or α-phosphothreonine antibodies.

FIG. 25 is a graph showing the effect of a ΔprkC mutation on B. anthracis spore germination. B. anthracis Sterne wild type or JDB1930 (ΔprkC) spores were incubated in the presence of 100 μg/ml B. anthracis peptidoglycan and % germination was determined. Error bars represent s.d. for triplicate samples.

FIG. 26 is a graph showing complementation of the ΔprkC_{K40A} mutation. Spores generated from strains JDB3 (PY79, wild type), PB705 (ΔprkC) JDB2227 (ΔprkC amyE::P_{spac}-FLAG-prkC_{Bs}) and JDB2228 (ΔprkC amyE::P_{spac}-FLAG-prkC_{Bs(K40A)}) were exposed to 1 mM L-alanine (ala), 100 μg/ml PG (PG) or 20 μM disaccharide tripeptide (tri) for 60 min prior to measuring % germination.

FIG. 27 is western blots showing spore fractionation. JDB2228 (ΔprkC amyE::P_{spac}-FLAG-prkC_{Bs(K40A)}), JDB1568 (cotE-gfp), and JDB1700 (Pₛₚₐₙₖ-gfp) spores were fractionated according to the protocol described for the localization for FLAG-PrkC. Detection of Flag-PrkC_{(K40A)} in the P100 fraction (IM) using α-FLAG antibodies, CotE-GFP in the coat fraction (C) and GFP in the S100 fraction (S) by α-GFP antibodies (kind gift from H. Shuman) is shown.

FIG. 28 is diagrams, a western blot and a graph showing localization and peptidoglycan binding of PrkC. Panel A is a schematic of PrkC localization. The DNA is located in the core and is surrounded by the cortex and the coat. PrkC is associated with the inner membrane (black) of the spore. Panel B shows a western blot when lysates of wild-type (PY79), ΔprkC (PB705), and ΔprkC amyE::P_{spac}-FLAG-prkC_{Bs}(JDB2226) spores were electrophoresed using 8% SDS-PAGE, and blots were probed with anti-FLAG antibody (Sigma). Whole cell lysate from wild-type spores (WT); whole cell lysate from ΔprkC spores (ΔprkC); coat fraction from JDB2226 (C); soluble S100 fraction from JDB2226 (S); insoluble P100 fraction from JDB2226 (IM). In Panel C, 50 μg of His-tagged extracellular domains of PrkC, Yycl or AcmA were incubated with ˜5 mg purified cell wall peptidoglycan. Centrifugation was used to separate protein bound to insoluble PG from unbound protein. Bound protein was eluted by subjecting insoluble fraction to 2% SDS. Fractions containing unbound protein, and protein remaining bound to insoluble PG were subjected to 8% SDS-PAGE and Coomassie blue staining and protein bands were quantified using Image J (NIH). The total protein that was incubated was normalized to 100% for unbound+bound and relative bound protein levels were calculated.

FIG. 29 is graphs showing substrate specificity of PrkC. In Panel A, JDB1980 (ΔprkC amyE::P_{spac}-his₆-prkC_{Bs}) or JDB2017 (ΔprkC amyE::P_{spac}-his₆-prkC_{Sa}) spores were incubated with different amounts of S. aureus PG for 60 min. In Panel B, 50 μg His₆-PASTA_{Bs} (PrkC_{Bs}) and His₆-PASTA_{Sa} (PrkC_{Sa}) were incubated with ˜5 mg S. aureus PG. Unbound proteins and bound proteins were detected by Coomassie blue and % bound protein was calculated as above.

FIG. 30 is a graph showing germination by S. aureus cell-free supernatant. JDB1980 (ΔprkC amyE::P_{spac}-his₆-prkC_{Bs}) or JDB2017 (ΔprkC amyE::P_{spac}-his₆-prkC_{Sa}) spores were incubated with S. aureus cell-free supernatant at a series of dilutions. Error bars represent s.d. for triplicate samples.

FIG. 31 is a graph showing regulation of germination by small molecules. Panel A shows % germination when wild type (squares) or ΔprkC (circles) B. subtilis spores were incubated for 60 min with bryostatin at indicated concentrations. Panel B shows % germination when wild type spores were incubated for 60 min with 100 ng/ml B. subtilis peptidoglycan in the presence of staurosporine at indicated concentrations. Panel C shows % germination when wild type spores were incubated for 60 min with B. subtilis peptidoglycan at the indicated concentrations in the presence or absence of 10 pM staurosporine. Error bars represent s.d. for triplicate samples.

FIG. 32 is a cartoon showing PrkC as a substrate of PrpC phosphatase in vivo.

FIG. 33 is a schematic diagram showing germination of wild type (WT) or ΔprpC mutants. Lack of PrpC phosphatase did not change the response to PG.

FIG. 34 is a bar graph showing percent germination of wild type (wt) and ΔprkC B. subtilis (ΔprkC) incubated with 500 μM teleocidin (indolactam). Teleocidin stimulated germination of wildtype spores.

### DETAILED DESCRIPTION OF THE INVENTION

The present application is based in part on the discovery that cell wall materials of bacteria stimulate germination of spores of Gram-positive bacteria. This stimulation of spore germination requires the activity of the Ser/Thr kinase PrkC, which appears to mediate the germination signal in the spore.

Bacteria can respond to adverse environmental conditions by drastically reducing or even ceasing metabolic activity. They must then determine that conditions have improved before exiting dormancy. One indication of such a change is the growth of other bacteria in the local environment. Growing bacteria release muropeptide fragments of the cell wall into the extracellular milieu. It is reported herein that these muropeptides are potent germinants of dormant Gram-positive bacteria spores, such as Bacillus subtilis spores. The ability of a muropeptide to act as a strong germinant can be determined by the identity of a single amino acid. As described herein, a well conserved, eukaryotic-like Ser/Thr membrane kinase containing an extracellular domain capable of binding peptidoglycan is necessary for this response and a small molecule that stimulates related eukaryotic kinases can be sufficient to induce germination. Furthermore, small molecule kinase inhibitors, such as staurosporine, can block muropeptide-dependent germination of dormant spores.

Provided herein are methods of stimulating germination of spores of Gram-positive bacteria, methods of inhibiting germination of spores of Gram-positive bacteria, compositions for the stimulation or inhibition of germination of spores of Gram-positive bacteria, methods of therapeutic treatment, methods of decontamination, methods of screening for stimulators or inhibitors of germination of spores of Gram-positive bacteria, and transgenic bacterial cells. Each such aspect is described further below.

Stimulating Germination

Provided is a method of stimulating germination of a spore of a first Gram-positive bacterium. Such methods are expected to be useful to stimulate germination of any spore-forming Gram-positive first bacterium. Various embodiments of the method comprise contacting the spore with (i) a peptidoglycan fragment or muropeptide of a second bacterium, such as a second Gram-positive bacteria or a second Gram-negative bacteria, or a preparation comprising cell walls from the second bacterium; or (ii) a compound that stimulates activity of a serine/threonine protein kinase of the first Gram-positive bacterium; or (iii) a compound that inhibits activity of a PPM-like phosphatase of the first Gram-positive bacterium.

The use of “or” in these methods does not exclude more than one of the options utilized in the method, since the word “comprises” has its usual open-ended meaning.

In some embodiments, spores of the first Gram-positive bacterium are stimulated to germinate by contacting the spore with a compound that stimulates activity of a serine/threonine protein kinase of the Gram-positive bacterium. As used herein, a serine/threonine protein kinase is an enzyme that catalyzes the phosphorylation of the OH group of a serine or a threonine residue in a protein.

In some embodiments, spores of the first Gram-positive bacterium are stimulated to germinate by contacting the spore with a peptidoglycan fragment of a second bacterium, such as a second Gram-positive bacteria or a second Gram-negative bacteria. In some embodiments, spores of the first Gram-positive bacterium are stimulated to germinate by contacting the spore with a muropeptide of a second bacterium, such as a second Gram-positive bacteria or a second Gram-negative bacteria. In some embodiments, spores of the first Gram-positive bacterium are stimulated to germinate by contacting the spore with a preparation comprising cell walls from the second bacterium, such as a second Gram-positive bacteria or a second Gram-negative bacteria.

In some embodiments, spores of the first Gram-positive bacterium are stimulated to germinate by contacting the spore with a compound that inhibits activity of a PPM-like phosphatase of the first Gram-positive bacterium.

Inhibiting Germination

Also provided is a method of inhibiting germination of a spore of a Gram-positive bacterium. In various embodiments, the method comprises contacting the spore with (i) a compound that inhibits activity of a serine/threonine protein kinase of a Gram-positive bacterium; or (ii) a compound that stimulates activity of a PPM-like phosphatase of the first Gram-positive bacterium.

In some embodiments, the spore is contacted with a compound that inhibits activity of a serine/threonine protein kinase of the Gram-positive bacterium.

Bacterial Serine/Threonine Protein Kinase

As described herein, muropeptide cell wall fragments are potent germinants of dormant Gram-positive bacteria spores, where germinant activity is correlated to the identity of a single amino acid of the muropeptide. As described herein, bacterial PrkC, a well conserved, eukaryotic-like Ser/Thr membrane kinase containing an extracellular domain capable of binding peptidoglycan, is necessary for this response.

Although phosphorylation of target proteins plays a central regulatory mechanism in the physiology of both bacterial cells and eukaryotic cells, it has been thought that the kinases responsible for these modifications are not homologous in either primary sequence or structure. Recent work, however, has identified a family of proteins in bacterial cells with substantial homology to eukaryotic Ser/Thr kinases (including the PrkC protein described herein) at both the sequence and structural level. These proteins have been shown to phosphorylate a number of substrates in bacterial cells on Serine and/or Threonine residues and mutagenic studies have indicated that they use a catalytic mechanism very similar to their eukaryotic counterparts. It is emphasized that there are other Ser/Thr kinases in bacterial cells, but these proteins do not appear to have significant homology at any level with any eukaryotic proteins.

In various embodiments, the PrkC comprises an amino acid sequence at least about 25% identical to the sequence encoded by the complement of nucleotides 2106-4033 of SEQ ID NO:1, which is expected to encompass any PrkC from a Clostridium or Bacillus species. The serine/threonine protein kinase of the Gram-positive bacterium can also be a PrkC comprising an amino acid sequence at least about 40% identical to the sequence encoded by the complement of nucleotides 2106-4033 of SEQ ID NO:1., which is expected to encompass any PrkC from a Bacillus sp. Additionally, the serine/threonine protein kinase of the Gram-positive bacterium can be a PrkC comprising an amino acid sequence at least about 50%, at least about 60%, at least about 70%, at least about 80%, at least about 90%, at least about 95%, at least about 99%, or completely identical to the sequence encoded by the complement of nucleotides 2106-4033 of SEQ ID NO:1.

In various embodiments, the PrkC comprises an amino acid encoded by a nucleic acid sequence that hybridizes under highly stringent hybridization conditions to a sequence comprising nucleotides 2106-4033 of SEQ ID NO:1, or a complement thereof.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ, of a DNA:DNA hybrid is decreased by 1-1.5° C. for every 1% decrease in nucleotide identity (see e.g., Sambrook and Russel, 2006).

Stimulator of Serine/Threonine Protein Kinase

Various embodiments described herein use a stimulator of serine/threonine protein kinase. As described herein, a well conserved, eukaryotic-like Ser/Thr membrane kinase containing an extracellular domain capable of binding peptidoglycan is necessary for cell wall muropeptide induction of Gram-positive bacteria spore germination, and a small molecule that stimulates related eukaryotic kinases can be sufficient to induce germination. Methods or compositions described herein are not limited to the use of any particular stimulant of a serine/threonine protein kinase and can include kinase stimulators that have not yet been discovered.

In some embodiments, the stimulator of serine/threonine protein kinase is a stimulator of PrkC protein.

Examples of a stimulator of serine/threonine protein kinase include, but are not limited to a phorbol ester, bryostatin, teleocidin, and phorbol-12-myristate-13-acetate (PMA). In some embodiments, the stimulator is a phorbol ester. In some embodiments, the stimulator is bryostatin. In some embodiments, the stimulator is teleocidin. In some embodiments, the stimulator is phorbol-12-myristate-13-acetate (PMA).

A compound for stimulating a serine/threonine protein kinase can be in a cell free supernatant of a bacterial extract. In some embodiments, the spore is contacted with a compound for stimulating a serine/threonine protein kinase compound by contacting the cell free supernatant.

Kinase Inhibitor

Various methods described herein use a kinase inhibitor. As described herein, small molecule kinase inhibitors can block muropeptide-dependent germination of dormant spores. Such methods are not limited to any particular inhibitor of the kinase and can include kinase inhibitors that have not yet been discovered.

In some embodiments, the serine/threonine protein kinase of the Gram-positive bacterium is a protein kinase C (PrkC), as described herein.

Examples of inhibitors that are expected to be useful for these methods include, but are not limited to: adaphostin, AG 490, AG 825, AG 957, AG 1024, aloisine, aloisine A, alsterpaullone, aminogenistein, API-2, apigenin, arctigenin, AY-22989, bisindolylmaleimide IX, chelerythrine, DMPQ, DRB, edelfosine, erbstatin analog, ET18OCH3, ERK inhibitor fasudil, gefitinib, H-7, H-8, H-89, HA-100, HA-1004, HA-1077, HA-1100, heatstable protein kinase A inhibitor PKI, hydroxyfasudil, indirubin-3′-oxime, 5-lodotubercidin, kenpaullone, KN-62, KY12420, LFM-A13, luteolin, LY-294002, LY294002, mallotoxin, ML-9, NSC-154020, NSC-226080, NSC-231634, NSC-664704, NSC-680410, NU6102, olomoucine, oxindole I, PD 153035, PD 98059, PD 169316, phloridzin, piceatannol, picropodophyllin, PKI, PP1, PP2, purvalanol A, quercetin, RAPA, rapamune, rapamycin, Ro 31-8220, roscovitine, rottlerin, SB202190, SB203580, sirolimus, SL327, SP600125, staurosporine, STI-571, SU1498, SU4312, SU6656, syk inhibitor, TBB, TCN, Triciribine, Tyrphostin AG 490, Tyrphostin AG 825, Tyrphostin AG 957, Tyrphostin AG 1024, U0126, W-7, wortmannin, Y-27632, ZD 1839, and ZM 252868. More specifically, the compound can be H-89, HA-1004, H-7, H-8, HA-100, PKI, bisindolylmaleimide IX, chelerythrine, edelfosine, edelfosina, ET18OCH3, H-7, HA-100, H89, HA-1004, Ro 31-8220, rottlerin, staurosporine, quercetin, Triciribine, KN-62, W-7, HA-1004, HA-1077, SB202190, SB203580, arctigenin, PD 98059, SL327, or U0126, which includes compounds that are specific inhibitors of serine/threonine protein kinase. In more specific embodiments, the compound is staurosporine.

Spore Location

Various embodiments involve modulation of germination of a spore of a Gram-positive bacteria. Such spore can be in vitro (e.g., an environmental contaminant) or in vivo (e.g., an infection in an animal).

When the spore is an environmental contaminant, the methods are not limited to any particular source of the contamination, and encompasses, e.g., spores that are from a saprophytic bacterial growth on any substrate (e.g., on food or animal feed). The spore can also be the product of a natural infection (e.g., on the skin of a slaughtered animal that had a natural infection, or emitted from a bacterial lesion from a human infection) or a deliberate contamination (e.g., a terrorist attack).

A spore located in vivo can be in an animal of any species, including birds. As used herein, the phrase “the spore is in” an animal, mammal, etc., includes spores that are on the surface of the animal or mammal, for example, as part of an infection or a saprophytic colonization of the skin or fur. In some embodiments the animal is a mammal, including any domesticated mammal and humans. These embodiments are not limited to any particular mammals and include domesticated mammals. Included here are bred rodents such as mice, rats, guinea pigs, and gerbils; dogs; cats; sheep; cows; horses; pigs; goats; donkeys and mules; and primates such as monkeys, prosimians, or apes. The mammal can also be a human.

Methods described herein can be performed on a subject in need thereof. A subject in need of the therapeutic methods described herein can be diagnosed with a Gram-positive bacterial infection, or at risk thereof. A determination of the need for treatment will typically be assessed by a history and physical exam consistent with the disease or condition at issue. Diagnosis of the various conditions treatable by the methods described herein is within the skill of the art.

Bacteria

Various methods described herein involve a Gram-positive bacterium. For example, described herein are methods for stimulating spore germination of a Gram-positive bacteria. As another example, described herein are methods for inhibiting spore germination of a Gram-positive bacteria. As another example, a cell wall preparation or muropeptides from a second Gram-positive bacteria are used to stimulate spore germination of a first Gram-positive bacterium. As another example, a cell wall preparation or muropeptides from a second Gram-negative bacteria are used to stimulate spore germination of a first Gram-positive bacterium.

In various embodiments, a Gram-positive bacteria can be selected from a Bacillus, Clostridium, Desulfotomaculum, Sporolactobacillus, Sporosarcina, Thermoactinomyces, Listeria, Streptococcus, or Streptomyces. Examples of Gram-positive bacteria include, but are not limited to, B. anthracis, B. cereus, B. thuringiensis, C. difficile, C. botulinum, B. subtilis, B. megaterium, B. anthracis, C. acetobutylicum, L. monocytogenes, and Streptomyces coelicolor.

A bacteria as recited in various embodiments herein can be a Gram-positive bacteria, and can also exhibit a dormant phase, a stationary growth phase, a cyst (e.g., exospore) stage, or a spore (e.g., endospore) stage. Examples of a bacteria with an endospore stage include, but are not limited to, Bacillus, Clostridium, Desulfotomaculum, Sporolactobacillus, Sporosarcina, and Thermoactinomyces. In some embodiments, the bacteria is a Bacillus sp. or a Clostridium sp. Examples of Bacillus sp. or a Clostridium sp. include, but are not limited to, B. anthracis, B. cereus, B. thuringiensis, C. difficile, C. botulinum, B. subtilis, B. megaterium, B. anthracis, and C. acetobutylicum.

In some embodiments, a Gram-positive bacterium is B. anthracis, B. cereus, B. thuringiensis, C. difficile, or C. botulinum. In some embodiments, a Gram-positive bacterium is B. subtilis, B. megaterium, B. anthracis, or C. acetobutylicum.

Various methods described herein recite a first Gram-positive bacterium and a second bacterium, such as a second Gram-positive bacterium or a second Gram-negative bacterium. In some embodiments, spore germination of the first Gram-positive bacterium is inhibited or stimulated. In some embodiments, a cell wall preparation from a second bacterium, such as a second Gram-positive bacterium or a second Gram-negative bacterium, is used to stimulate germination of a first Gram-positive bacterium.

A first Gram-positive bacterium can include any of the Gram-positive bacteria discussed above. In some embodiments, a first Gram-positive bacterium is a Bacillus sp. or a Clostridium sp. Examples of a first Gram-positive bacterium include, but are not limited to, B. anthracis, B. cereus, C. difficile, C. botulinum, or B. thuringiensis. In some embodiments, the first Gram-positive bacterium is B. anthracis, B. cereus, B. thuringiensis, C. difficile, or C. botulinum. In some embodiments, the first Gram-positive bacterium is B. anthracis, B. cereus, C. difficile, or C. botulinum.

A second bacterium, the cell wall materials of which can be used to stimulate germination of a first Gram-positive bacteria, can be a second Gram-positive bacteria or a second Gram-negative bacteria. A second Gram-negative bacterium can include E. coli (see Example 8). A second Gram-positive bacterium can include any of the Gram-positive bacteria discussed above. In some embodiments, the second Gram-positive bacterium is a Bacillus, Clostridium, Listeria, or Streptomyces. For example, the second Gram-positive bacterium can be B. subtilis, B. megaterium, B. anthracis, C. acetobutylicum, L. monocytogenes, or S. coelicolor. The second Gram-positive bacterium can be of the same genus as the first Gram-positive bacterium. The second Gram-positive bacterium can be of a different genus as the first Gram-positive bacterium. The second Gram-positive bacterium can be of the same species as the first Gram-positive bacterium. The second Gram-positive bacterium can be of a different species as the first Gram-positive bacterium.

Various methods described herein are not limited to any particular source of a first Gram-positive bacterium. Various methods described herein are not limited to any particular source of a second bacterium, whether Gram-positive or Gram-negative. As discussed in the Examples below, there can be some species specificity as to the relationship between a first Gram-positive bacteria and a second Gram-positive bacteria. It is expected that cell walls of any species of Bacillus or Clostridium can stimulate germination of spores of any other species of those genera. In some embodiments, a second bacterium comprises a peptidoglycan containing m-Dpm at the third position, which is correlated with strong germinant activity (see e.g., Table 3). Examples of Gram-positive bacterium comprising a peptidoglycan containing m-Dpm at the third position include, but are not limited to, B. subtilis, B. megaterium, B. anthracis, C. acetobutylicum, L. monocytogenes, and S. coelicolor.

Peptidoglycan Fragment, Muropeptide, and Cell Wall Preparation

Various embodiments involve peptidoglycan fragments, muropeptides, and cell wall preparations of or from a second bacterium, such as a second Gram-positive bacterium or a second Gram-negative bacterium. Methods described herein are not limited to any particular source of the second bacterium. As discussed in the Examples below, there is some species specificity as to the relationship between a first and a second bacteria.

In some embodiments, the peptidoglycan fragments, muropeptides, or cell wall preparations comprise a peptidoglycan containing m-Dpm at the third position, which is correlated with strong germinant activity (see e.g., Table 3). In various embodiments, a cell wall preparation is derived from a second Gram-positive bacterium, examples of which are provided above. It is expected that cell walls of any species of Bacillus or Clostridium can stimulate germination of spores of any other species of those genera. In various embodiments, a cell wall preparation is derived from a second Gram-negative bacterium, examples of which are provided above.

The peptidoglycan fragments, muropeptides, or cell wall preparations can be at any level of purification. Peptidoglycan fragments can comprise synthetic peptidoglycan fragments, naturally occurring peptidoglycan fragments derived from a bacteria (e.g., a Gram-positive bacterium or a Gram-negative bacterium), or a combination thereof. Muropeptides can comprise synthetic muropeptides, naturally occurring muropeptides derived from a bacteria (e.g., a Gram-positive bacterium or a Gram-negative bacterium), or a combination thereof. The cell walls from the second bacterium (e.g., a Gram-positive bacterium or a Gram-negative bacterium) can be a crude preparation (e.g., a whole cell preparation). The cell wall preparation can comprise purified peptidoglycan fragments or muropeptides. An included preparation here is purified natural or synthetic peptidoglycan fragments or muropeptides. In some embodiments, the peptidoglycan fragments or muropeptides comprise a diaminopimelic acid (DAP) in the third residue of the stem-peptide (see Table 3). The cell wall preparation can be a supernatant fraction from growing cells, which contains cell wall fragments (see Examples). In some embodiments, the preparation of cell walls does not contain a living second bacterium (e.g., a Gram-positive bacterium or a Gram-negative bacterium).

Compositions

The application is further directed to a composition comprising an antibiotic and (i) a preparation of cell walls from a second bacterium, such as a Gram-positive or Gram-negative bacterium or (ii) a compound that stimulates activity of a serine/threonine protein kinase of a Gram-positive bacterium or (iii) a compound that inhibits activity of a PPM-like phosphatase of a Gram-positive bacterium, in a pharmaceutically acceptable carrier or excipient. In some embodiments, the antibiotic is a broad spectrum antibiotic. In some embodiments, the antibiotic is effective against a Gram-positive bacterium. These compositions can be useful for, e.g., treating an animal, such as a mammal, infected with a spore forming Gram-positive bacterium, where the cell walls or compound stimulates germination of spores, making them more susceptible to the antibiotic.

An antibiotic in these compositions can be effective against a Gram-positive bacterium. The antibiotic for such compositions can include any antibiotic, now known or later discovered, that is effective against the Gram-positive bacterium. Examples of such antibiotics include, but are not limited to, a beta-lactam, clavulanic acid, a monobactam, a carboxypenem, an aminoglycoside, gentamicin, a glycopeptide, a lincomycin, a macrolide, bacitracin, a rifamycin, a tetracycline, or chloramphenicol. More specifically, suitable antibiotics for various embodiments include penicillin G, benzathine benzylpenicillin, phenoxymethylpenicillin, amoxicillin, ampicillin, bacampicillin, pivampicillin, clavulanic acid, aztreonam, imipenem, streptomycin, gentamicin, vancomycin, clindamycin, erythromycin, bacitracin, rifampicin, tetracycline, doxycycline, tigecycline, chloramphenicol, linezolid, quinupristin-dalfopristin, and daptomycin.

In some embodiments, the composition comprises an antibiotic effective against a Gram-positive bacterium and a preparation of cell walls from a bacterium, such as a Gram-positive bacterium or a Gram-negative bacterium. A preparation of cell walls from a bacterium can be as described above.

In some embodiments, the composition comprises an antibiotic effective against a Gram-positive bacterium and a compound that stimulates activity of a serine/threonine protein kinase of a Gram-positive bacterium. For example, the composition can comprise a stimulator of PrkC. The compound that stimulates activity of a serine/threonine protein kinase of a Gram-positive bacterium can be as described above.

In some embodiments, the composition comprises an antibiotic effective against a Gram-positive bacterium and a compound that inhibits activity of a PPM-like phosphatase of a Gram-positive bacterium. The compound that inhibits activity of a PPM-like phosphatase of a Gram-positive bacterium can be as described above.

Agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, e.g., in a purified form, together with a suitable amount of carrier so as to provide the form for proper administration to the subject.

The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents and compositions described herein (e.g., agents and compositions that modulate activity of a serine/threonine protein kinase of a Gram-positive bacterium) can also be formulated or used in combination with other therapeutic modalities. Thus, in addition to the therapies described herein, one may also provide to the subject other therapies known to be efficacious for bacterial infection.

Therapeutic Methods

Also provided is a method of treating a subject, such as a mammal, in need thereof. For example, a subject infected with a spore-forming Gram-positive bacterium can be treated according to methods described herein. In some embodiments, the method comprises administering a composition described herein to the subject.

Growing bacteria release muropeptide fragments of the cell wall into the extracellular milieu. It is reported herein that these muropeptides are potent germinants of dormant Gram-positive bacteria spores, such as Bacillus subtilis spores. The ability of a muropeptide to act as a strong germinant can be determined by the identity of a single amino acid. As described herein, a well conserved, eukaryotic-like Ser/Thr membrane kinase containing an extracellular domain capable of binding peptidoglycan can play a role in this response and a small molecule that stimulates related eukaryotic kinases can be sufficient to induce germination.

Methods described herein can be used to treat a subject infected with a spore-forming Gram-positive bacterium where the spore, but not the vegetative cell, is resistant to an antibiotic. A cell wall preparation, kinase stimulator, PPM-like phosphatase inhibitor, or combination thereof can induce germination of the spore, allowing antibiotic killing, thus preventing the pathogen from escaping the antibiotic.

In some embodiments where stimulation of spore germination is desired, the method can comprise administering to a subject in need thereof an antibiotic and (i) a preparation of cell walls from a second bacterium, such as a Gram-positive bacterium or a Gram-negative bacterium; (ii) a compound that stimulates activity of a serine/threonine protein kinase of the Gram-positive bacterium; (iii) a compound that inhibits activity of a PPM-like phosphatase of a Gram-positive bacterium; or a combination thereof. In some embodiments where stimulation of spore germination is desired, the method can comprise administering to a subject in need thereof (i) a preparation of cell walls from a second bacterium, such as a Gram-positive bacterium or a Gram-negative bacterium; (ii) a compound that stimulates activity of a serine/threonine protein kinase of the Gram-positive bacterium; (iii) a compound that inhibits activity of a PPM-like phosphatase of a Gram-positive bacterium; or a combination thereof. An effective amount of a such composition(s) is generally that which can induce the germination of a Gram-positive bacteria spore, kill germinant or vegetative bacteria cells, or a combination thereof.

For example, the method can comprise administering an antibiotic and a preparation of cell walls from a Gram-positive bacterium. As another example, the method can comprise administering an antibiotic and a preparation of cell walls from a Gram-negative bacterium. As another example, the method can comprise administering an antibiotic and a compound that stimulates activity of a serine/threonine protein kinase of a Gram-positive bacterium. As another example, the method can comprise administering an antibiotic and a compound that inhibits activity of a PPM-like phosphatase of a Gram-positive bacterium. The antibiotic and the agent(s) of (i), (ii), or (iii) can be administered as separate agents or combined in a single composition. The agents of (i), (ii), or (iii) can be administered as separate agents or combined in a single composition.

In some embodiments where inhibition of spore germination is desired, the method can comprise administering to a subject in need thereof (i) a compound that inhibits activity of a serine/threonine protein kinase of a Gram-positive bacterium or (ii) a compound that stimulates activity of a PPM-like phosphatase of a Gram-positive bacterium. The agents of (i) or (ii) can be administered as separate agents or combined in a single composition. An effective amount of a such composition(s) is generally that which can inhibit the germination of a Gram-positive bacteria spore. Such methods can be useful to prevent a spore from germinating and causing disease in a subject. These methods can further comprise administering an antibiotic (in the same or a different composition) that is effective against the Gram-positive bacterium to the subject, in order to kill some or all antibiotic-susceptible vegetative cells present. An effective amount of a such composition(s) is generally that which can inhibit the germination of a Gram-positive bacteria spore, kill vegetative bacteria cells, or a combination thereof.

Methods described herein can be performed on a subject in need thereof. A subject in need of the therapeutic methods described herein can be diagnosed with a Gram-positive bacterial infection, or at risk thereof. A bacterial infection treatable according to methods described herein can be an infection by a Gram-positive bacterium as recited in the context of a first Gram-positive bacterium described above. For example, the Gram-positive bacterium can be a Bacillus sp. or a Clostridium sp (e.g., B. anthracis, B. cereus, C. difficile, or C. botulinum). A determination of the need for treatment will typically be assessed by a history and physical exam consistent with the disease or condition at issue. Diagnosis of the various conditions treatable by the methods described herein is within the skill of the art.

A subject treated according to methods described herein can be an animal of any species, including birds. In some embodiments the animal is a mammal. These embodiments are not limited to any particular mammals and include domesticated mammals. Subjects treatable according to methods described herein include, but are not limited to, bred rodents such as mice, rats, guinea pigs, and gerbils; dogs; cats; sheep; cows; horses; pigs; goats; donkeys and mules; primates such as monkeys, prosimians, or apes; and humans.

When used in the methods described herein, a therapeutically effective amount of a composition described herein can be employed in pure form or, where such forms exist, in pharmaceutically acceptable salt form and with or without a pharmaceutically acceptable excipient. For example, the compositions of the invention can be administered, at a reasonable benefit/risk ratio applicable to any medical treatment, in a sufficient amount to modulate the activity of a serine/threonine protein kinase of a Gram-positive bacterium.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of a composition described herein can occur as a single event or over a time course of treatment. For example, a composition can be administered daily, weekly, bi-weekly, or monthly. For treatment of acute conditions, the time course of treatment can be at least several days. Some conditions could extend treatment from several days to several weeks. For example, treatment could extend over one week, two weeks, or three weeks. For other conditions, treatment could extend from several weeks to several months or even a year or more.

Treatment in accord with the methods described herein can be performed prior to, concurrent with, or after conventional treatment modalities for a bacterial infection or conditions associated therewith.

A composition described herein can be administered simultaneously or sequentially with another agent, such as an antibiotic, an antiinflammatory, or another agent. For example, a composition for the stimulation of germination of a spore of a Gram-positive bacteria can be administered simultaneously with another agent, such as an antibiotic or an antiinflammatory. Simultaneous administration can occur through administration of separate compositions, each containing one or more of an agent or composition described herein, an antibiotic, an antiinflammatory, or another agent. Simultaneous administration can occur through administration of one composition containing two or more of an agent or composition described herein, an antibiotic, an antiinflammatory, or another agent. An agent or composition described herein can be administered sequentially with an antibiotic, an antiinflammatory, or another agent. For example, an agent or composition described herein can be administered before or after administration of an antibiotic, an antiinflammatory, or another agent.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the invention.

Delivery systems may include, for example, an infusion pump which may be used to administer the agent or composition in a manner similar to that used for delivering insulin or chemotherapy to specific organs or tumors. Typically, using such a system, an agent or composition is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Decontamination Methods

Also provided is a method of decontaminating an environment containing, or suspected of containing, spores of a first Gram-positive bacterium. In some embodiments, the method comprises treating the environment with (i) a preparation of cell walls from a second bacterium, such as a second Gram-positive bacterium or a second Gram-negative bacterium; (ii) a compound that stimulates activity of a serine/threonine protein kinase of a Gram-positive bacterium; or (iii) a compound that inhibits activity of a PPM-like phosphatase of a Gram-positive bacterium. Such a method can be useful to stimulate germination of a spore-forming Gram-positive first bacterium environmental contaminant. A first Gram-positive bacterium and a second bacterium can be as described above. The second Gram-positive bacterium can be of the same genus as the first Gram-positive bacterium environmental contaminant. The second Gram-positive bacterium can be of the same species as the first Gram-positive bacterium environmental contaminant.

Various embodiments of decontamination methods are not limited to any particular source of the contamination, and encompass, e.g., spores that are from a saprophytic bacterial growth on any substrate (e.g., on food or animal feed). The spore can also be the product of a natural infection (e.g., on the skin of a slaughtered animal that had a natural infection, or emitted from a bacterial lesion from a human infection) or a deliberate contamination (e.g., a terrorist attack). Non-limiting examples of environments that may be contaminated include a room where mail is handled, a hospital, and an animal skin from an animal that had an infection of the spore-forming Gram-positive bacterium.

Screening Methods

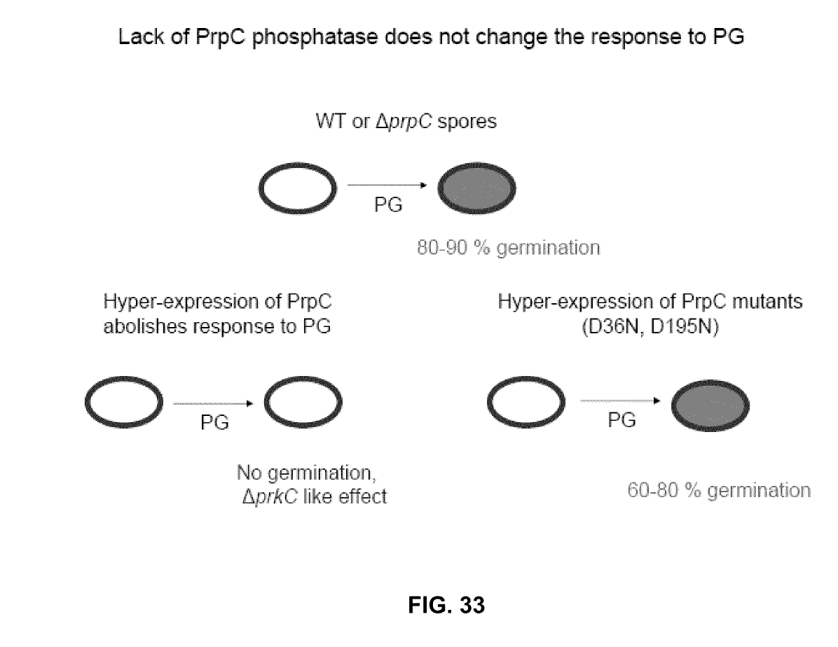

Also provided is a method to identify a compound that can block the ability of a bacteria spore to reactivate by inhibiting a Ser/Thr kinase, where such kinase activity is essential for reactivation. In some embodiments, the Ser/Thr kinase can be inhibited directly or by stimulating activity of a PPM-like phosphatase (see Example 18). Phosphatase activity can counter Ser/Thr kinase activity (see Example 18), and overexpression of phosphatase can block germination (see e.g., FIG. 33). Furthermore, phosphatase activity can be required for blocking germination, as shown by experiments wherein overexpression of mutant PrpC having single nucleotide polymorphisms were not effective in blocking germination as did overexpression of PrpC (see e.g., FIG. 33). These methods are also termed “assays” herein.

Also provided is a method of identifying a compound that can stimulate germination of a spore of a Gram-positive bacterium. In various embodiments, the method comprises determining whether the compound (i) stimulates activity of a serine/threonine protein kinase, or (ii) inhibits activity of a PPM-like phosphatase, of the Gram-positive bacterium. In these embodiments, a compound that stimulates activity of a serine/threonine protein kinase or inhibits activity of a PPM-like phosphatase of the Gram-positive bacterium stimulates germination of the spore of the Gram-positive bacterium.

Furthermore, various embodiments of the assay methodology herein provide a robust spore-based assay. Such an approach can avoid issues and problems associated with a cell-based assay. For example, with a spore-based assay, there is a greatly reduced need for maintaining living bacterial cells to be screened. By their very nature, the spores for use in the assay are robust. And the spore-based assay is an in vivo assay, which provides additional benefits over an in vitro assay.

In some embodiments, a compound is screened to determine whether the compound can inhibit germination of a spore of a Gram-positive bacterium. Such method can comprise determining whether the compound (i) inhibits activity of a serine/threonine protein kinase (e.g., PrkC), or (ii) stimulates activity of a PPM-like phosphatase (e.g., PrpC), of a Gram-positive bacterium. In these embodiments, inhibition of activity of a serine/threonine protein kinase or stimulation of activity of a PPM-like phosphatase of the Gram-positive bacterium can be correlated to an ability to inhibit germination of a spore of the Gram-positive bacterium.

In various embodiments, a candidate compound and a bacterial spore are combined, after which germination of the spore is monitored, Ser/Thr kinase activity is monitored, PPM-like phosphatase activity is monitored, or some combination thereof.

Some embodiments are directed to a system for screening candidate substances for actions on Mtb kinase, which can be useful for the development of compositions for therapeutic or prophylactic treatment of tuberculosis. Desirable properties of candidate substances include, but are not limited to, the ability to inhibit Mtb kinase, an essential component of Mtb reactivation.

An exemplary embodiment involves screening for inhibitors of Mycobacterium tuberculosis, a Ser/Thr kinase that is a tuberculosis drug target (Fernandez et al., 2006). Mtb kinase can be insensitive in vivo to some commercially available kinase inhibitors. Furthermore, there are numerous difficulties in using either in vitro or in vivo strategies to identify compounds that target Mtb kinase, including inability to assay bacterial cell permeability of compounds in an in vitro assay and the difficulty of working with Mtb in vivo, at least because of its about 8 hour replication time.

In some embodiments, a candidate substance for the treatment of tuberculosis can be screened by providing a bacterial spore stably expressing a Mycobacterium tuberculosis (Mtb) kinase in a suitable culture medium or buffer, administering the candidate substance to the spore, measuring the levels germination of the spore, and determining whether the candidate inhibits Mtb kinase activity of the spore. Alternatively, a candidate substance can be screened by providing a bacterial spore stably expressing a Mtb kinase in a suitable culture medium or buffer, administering the candidate substance to the cell, measuring the levels of germination of the spore, and determining whether the candidate substance decreases germination rates. Desirable candidates will generally possess the ability to inhibit Mtb kinase and decrease germination rates.

Other embodiments are directed to a system for screening candidate substances for actions on S. aureus kinase, which can be useful for the development of compositions for therapeutic or prophylactic treatment of bacterial infections highly resistant to antibiotics. Desirable properties of candidate substances include, but are not limited to, the ability to inhibit S. aureus kinase.

Further, methods described herein provide a way to identify compounds that can inhibit kinases that are relatively insensitive to staurosporine. The inventors have shown that the germination of spores expressing the B. subtilis kinase is sensitive to inhibition by the ATP analog staurosporine (˜pM). In contrast, spores expressing the Mtb kinase are less sensitive to inhibition by the ATP analog staurosporine (˜μM). Using an embodiment of a heterologous system described herein can provide for identification of inhibitors of Mtb kinases (or kinases from other bacteria, e.g., S. aureus kinase) showing relative insensitivity to staurosporine but without the necessity of screening Mtb (or S. aureus kinase) directly.

Compounds identified as having an effect on germination of bacterial spores carrying an exogenous kinase can then be more closely examined for their effect on the source bacteria of the exogenous kinase. Such an approach can overcome recognized problems for in vitro or in vivo screening of Mtb kinase or S. aureus kinase.

In other embodiments, germination of Gram-positive bacterial spores, such as from Bacillus or Clostridium, overexpressing a PPM-like phosphatase is monitored in the presence of candidate compounds, for example from small molecule kinase inhibitor libraries. Bacterial strains overexpressing a PPM-like phosphatase can be useful for evaluating the activity of potential agents on spore reactivation.

These screening methods can be performed on any spore-forming Gram-positive bacteria, including those described above (e.g., a first Gram-positive bacteria). For example, screening methods described herein can performed on spore-forming Gram-positive bacteria including, but not limited to a Bacillus sp. or a Clostridium sp., for example a B. anthracis, B. cereus, C. difficile, or C. botulinum.

The screened bacteria spore can be, for example, a Gram-positive bacteria as described above. In some embodiments, the screened bacteria is a transgenic bacteria expressing a heterologous Ser/Thr kinase or a PPM-like phosphatase. As an example, the screened bacteria can be a Bacillus expressing an Mtb Ser/Thr kinase or PPM-like phosphatase. As another example, the screened bacteria can be a Bacillus expressing a S. aureus Ser/Thr kinase or PPM-like phosphatase. As another example, the screened bacteria can be a Clostridium expressing an Mtb Ser/Thr kinase or PPM-like phosphatase. As another example, the screened bacteria can be a Clostridium expressing a S. aureus Ser/Thr kinase or PPM-like phosphatase. Such an approach can overcome recognized problems for in vitro or in vivo screening of Mtb or S. aureus kinase or phosphatase.

Any method suitable for detecting levels of Ser/Thr kinase or PPM-like phosphatase can be employed for levels resultant from administration of the candidate substance. Any method suitable for detecting germination rates of bacterial spores can be employed for levels resultant from administration of the candidate substance.

Monitoring of germination can be according to any method known in the art. For example, monitoring germination of a Gram-positive bacterial spore can be according to fluorescence changes or changes in heat resistance.

In some embodiments, monitoring germination of a Gram-positive bacterial spore can be according to changes in fluorescence. Monitoring of germination by changes in fluorescence can be performed on a time scale of about minutes, thus allowing high-throughput screening. To monitor germination according to changes in fluorescence, a bacterial spore and a fluorescent dye can be combined (see e.g., Example 5). In some embodiments, the fluorescent dye does not penetrate a nongerminating spore but does penetrate a spore undergoing reactivation or germination. The presence of a fluorescent dye within a bacterial spore or cell in these embodiments is an indicator of germination of the spore or cell. One example of a fluorescent dye that can be used to monitor bacterial spore germination is Syto-9 dye.

In some embodiments, monitoring germination of a Gram-positive bacterial spore can be according to changes in heat resistance (see e.g., Example 4). As an example, monitoring of germination can be according to methods disclosed in U.S. Pat. No. 6,596,496, incorporated herein by reference in its entirety.

Other methods of monitoring germination of bacterial spores are known in the art. An artisan of ordinary skill could determine an appropriate method of monitoring germination for any particular embodiment without undue experimentation.

Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals. In one embodiment, the candidate substance for screening is a small organic molecule. For the balance of the discussion, a candidate substance and a candidate molecule are used interchangeably and include at least all substances recited above.

Candidate molecules for screening according to methods disclosed herein also include those from small molecule libraries.

For example, a candidate molecule can be from a small molecule kinase inhibitor library. Candidate molecules for Ser/Thr kinase inhibitor screening according to methods disclosed herein include, but are not limited to, known inhibitors. As an example, the methods described herein can be used to screen kinase inhibitors and specific inhibitors of serine/threonine protein kinase, recited and described in further detail above.

As another example, candidate molecules for Ser/Thr kinase stimulator screening according to methods disclosed herein include, but are not limited to, known stimulators. As an example, the methods described herein can be used to screen inhibitors such as phorbol esters, bryostatins, teleocidin, or related compounds or derivatives thereof.

A candidate molecule can also be a modified version of the above molecules, e.g., designed to be more polar or better fitting to a receptor. In one embodiment, staurosporine-like compounds are screened for ability to inhibit Ser/Thr kinase and/or bacterial spore germination.

Transgenic Cell

Also provided is a transgenic bacteria expressing an exogenous Ser/Thr kinase or PPM-like phosphatase. Such a transgenic bacteria can be in accordance with those described above in the context of screening methods or assays.

In various embodiments, the host bacteria is a Gram-positive bacteria. The host bacteria can be any of the Gram-positive bacteria recited and discussed above. In some embodiments, the host bacteria is a Gram-positive bacteria, and can also exhibit a dormant phase, a stationary growth phase, a cyst (e.g., exospore) stage, or a spore (e.g., endospore) stage. Gram-positive bacteria with an endospore stage can be as recited and discussed above. In some embodiments, the host bacteria is a Bacillus, Clostridium, Desulfotomaculum, Sporolactobacillus, Sporosarcina, or Thermoactinomyces. Exemplary host bacteria include, but are not limited to, Bacillus sp. or a Clostridium sp, for example B. anthracis, B. cereus, B. thuringiensis, C. difficile, or C. botulinum.

The heterologous Ser/Thr kinase or PPM-like phosphatase can exhibit complementary action to a native Ser/Thr kinase or PPM-like phosphatase of the host. In various embodiments, a native Ser/Thr kinase or PPM-like phosphatase of the host is downregulated, silenced, or deleted. In some embodiments, the heterologous Ser/Thr kinase or PPM-like phosphatase is from a Gram-positive bacteria. More specifically, the heterologous Ser/Thr kinase or PPM-like phosphatase can be from a Gram-positive bacteria associated with a disease or condition, especially those Gram-positive bacteria difficult to culture and/or screen. The Ser/Thr kinase or PPM-like phosphatase to be inserted into a host can be for example from Mtb or S. aureus. In some embodiments, a host bacteria is transformed to express an Mtb Ser/Thr kinase (see e.g., Example 2) or PPM-like phosphatase. In other embodiments, a host bacteria is transformed to express a S. aureus Ser/Thr kinase (see e.g., Example 3) or PPM-like phosphatase.

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Methods for expressing proteins in prokaryotic hosts are well-known to those of skill in the art (see e.g., Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253). One skilled in the art can adapt known methods for expressing proteins in prokaryotic hosts so as to incorporate aspects of the present invention.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a preparation of cell walls from a second bacterium, such as a Gram-positive bacterium or a Gram-negative bacterium; a compound that stimulates activity of a serine/threonine protein kinase of a Gram-positive bacterium; a compound that inhibits activity of a PPM-like phosphatase of a Gram-positive bacterium; an antibiotic effective against a Gram-positive bacteria; or one or more compositions comprising such. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the invention are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Cell Wall as a Signal for Bacterial Growth

The cell wall provides structural integrity to bacterial cells. When Gram-positive cells grow, they release ˜50% of their cell wall material into the milieu. The response of spores to this material was investigated.

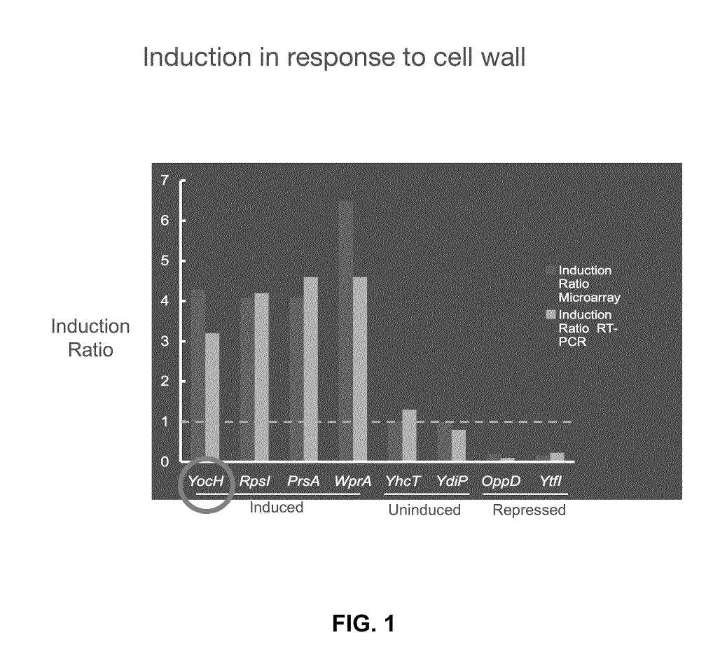

The response of Bacillus subtilis to exposure to cell wall fragments was studied by gene microarray analysis. A number of genes were induced and others were repressed (see e.g, FIG. 1). This observation was confirmed by RT-PCR. One of these genes, yocH, was further evaluated.

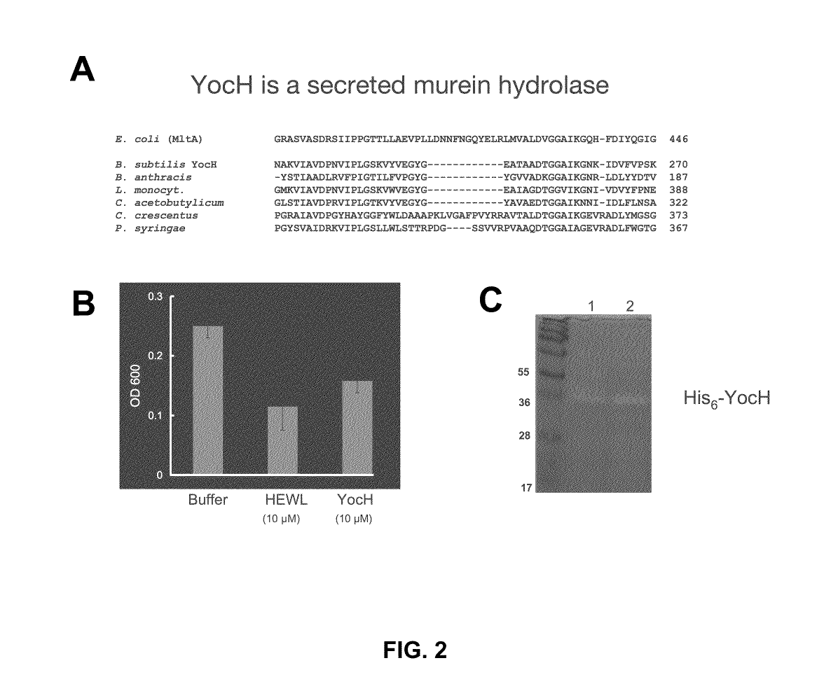

YocH belongs to a diverse family of bacterial proteins that are secreted and that share conserved aspartate residues with the MltA protein of E. coli that is known to have muralytic activity (see e.g, FIG. 2A). A His-tagged version of YocH was cloned and purified. YocA was then shown to lyse bacterial cells similar to hen egg white lysozyme (see e.g, FIG. 2B) and generate a clearance at the appropriate molecular weight in a zymogram (see e.g, FIG. 2C).

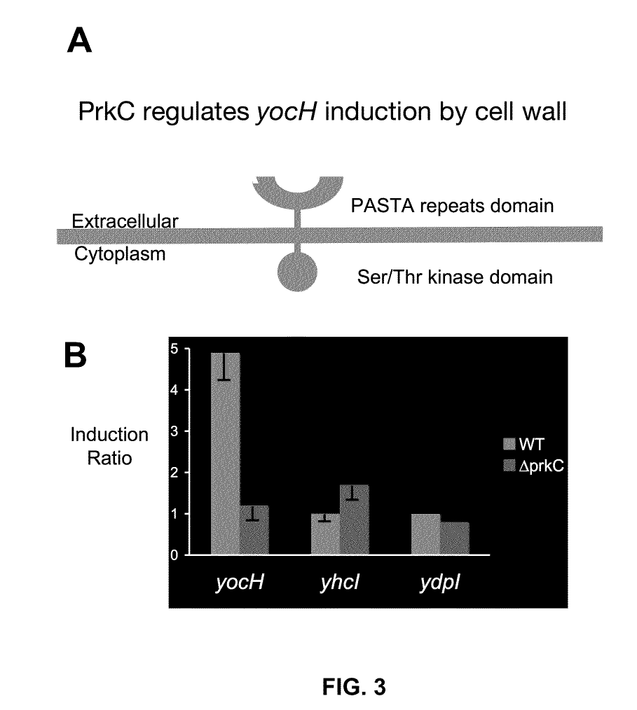

It was also determined that induction of yocH in response to exogenous cell wall was dependent on the PrkC Ser/Thr kinase, which is composed of an extracellular domain that has been hypothesized to bind peptidoglycan, a membrane spanning segment and an intracellular kinase domain (see e.g, FIG. 3).

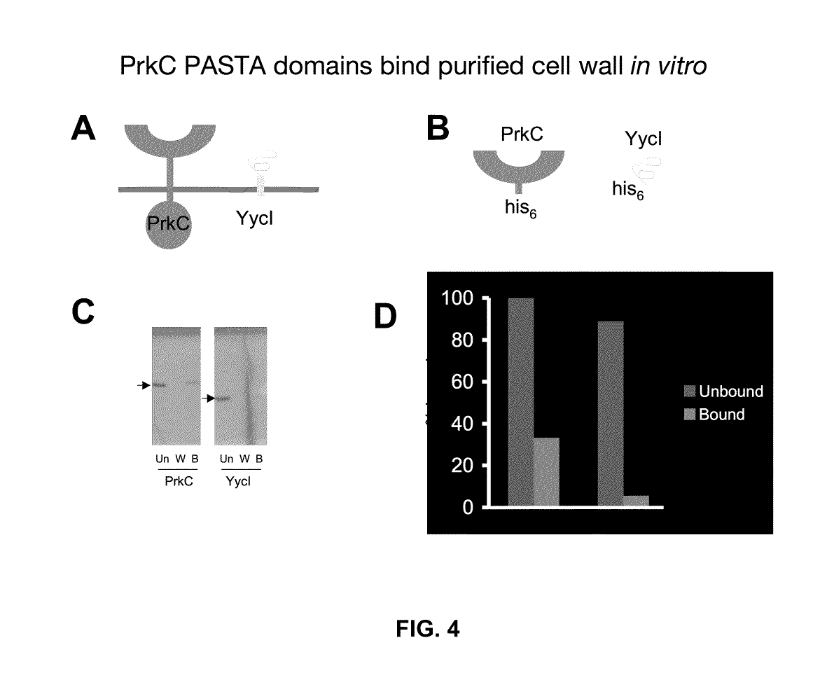

A truncated His-tagged protein containing only the extracellular domain was purified. This extracellular domain of PrkC bound cell wall itself, as demonstrated by showing that this protein bound to cell wall much better than a control protein (see e.g, FIG. 4).

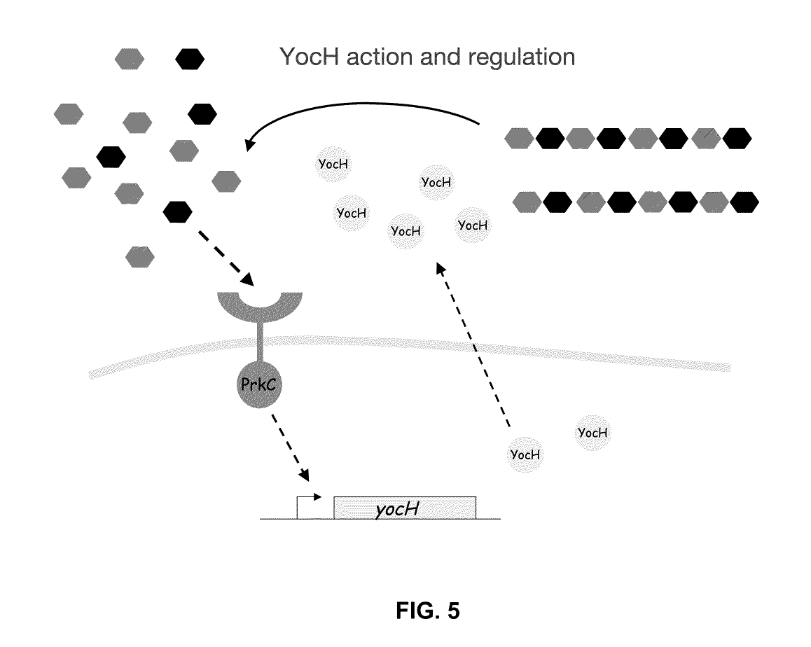

FIG. 5 shows a model developed based on these results. YocH is constitutively synthesized at a low level during growth, possibly due to the digestion of a small amount of extracellular peptidoglycan (PG) that bind to PrkC and stimulate its activity (and, indirectly, the expression of YocH). During stationary phase when there is an accumulation of cell wall material in the cellular milieu, YocH acts on this material, releasing a large amount of cell wall fragments, which bind to PrkC and greatly stimulate its activity and, indirectly, the expression of yocH.

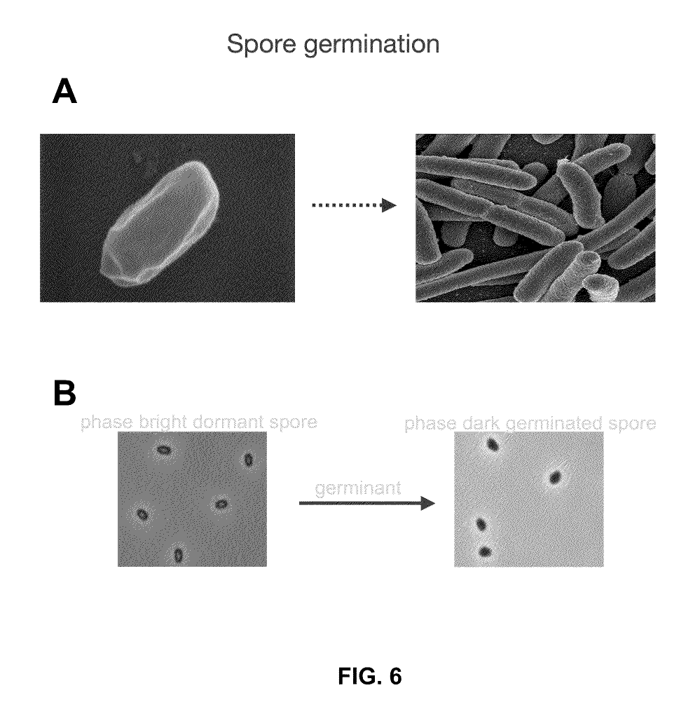

Spores are dormant, environmentally resistant forms of certain bacterial species. They can be induced to resume growth, i.e., to germinate (see e.g, FIG. 6A), by the addition of nutrients such as amino acids, but at non-physiologically relevant levels (e.g. >10 mM). The germinated spore can be readily distinguished from the ungerminated spore by phase contrast microscopy (see e.g, FIG. 6B). A signal that germination conditions are favorable was hypothesized to be the growth of neighboring cells. Those growing cells release a large amount of cell wall material into the milieu. Thus, this cell wall material (presumably fragments of some kind) would be an excellent signal for germination.

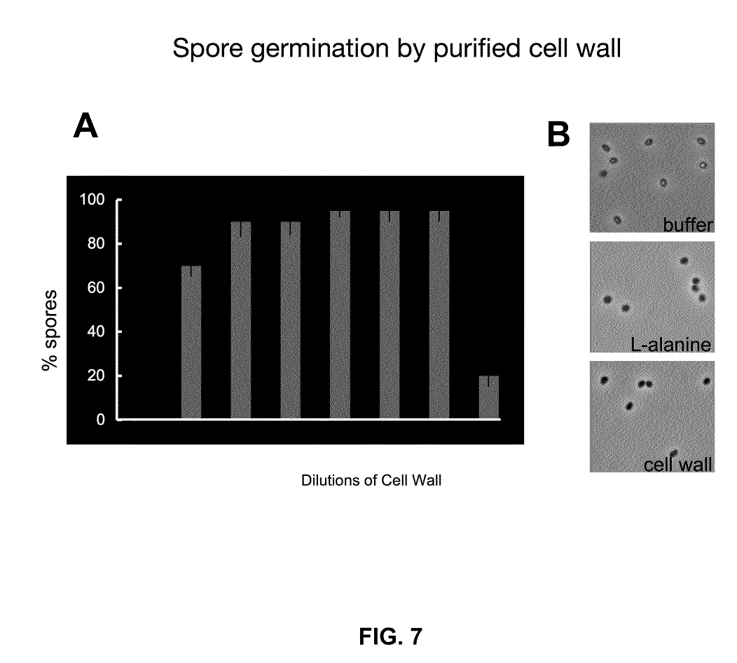

This hypothesis was tested by purifying cell wall from B. subtilis and adding it to spores. It was observed that this material worked very well, with amounts ˜1 μg apparently sufficient to germinate spores (see e.g, FIG. 7).

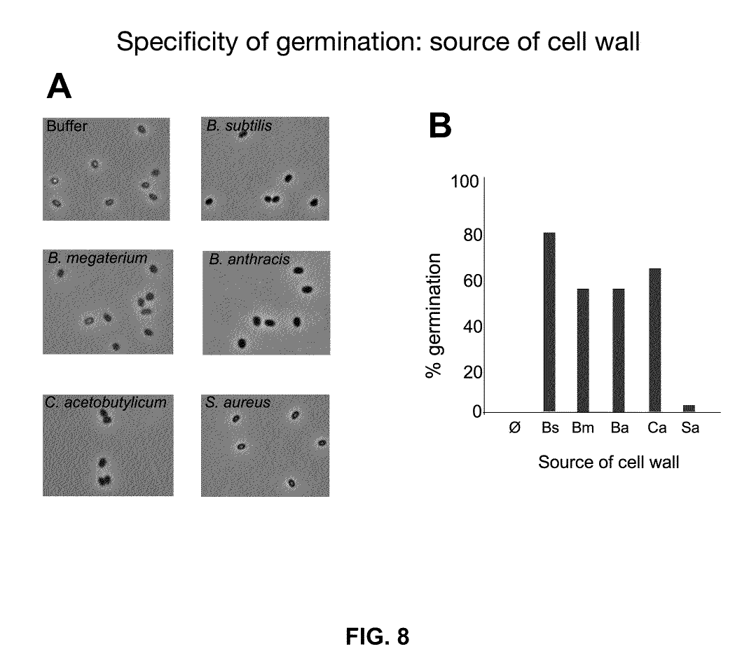

Cell wall purified from other spore-forming bacteria (e.g. B. anthracis, B. megaterium) also worked well; however, cell wall from other Gram-positive bacteria such as S. aureus did not appear to work (see e.g, FIG. 8). Thus, there appeared to be specificity in the germination response to cell wall.

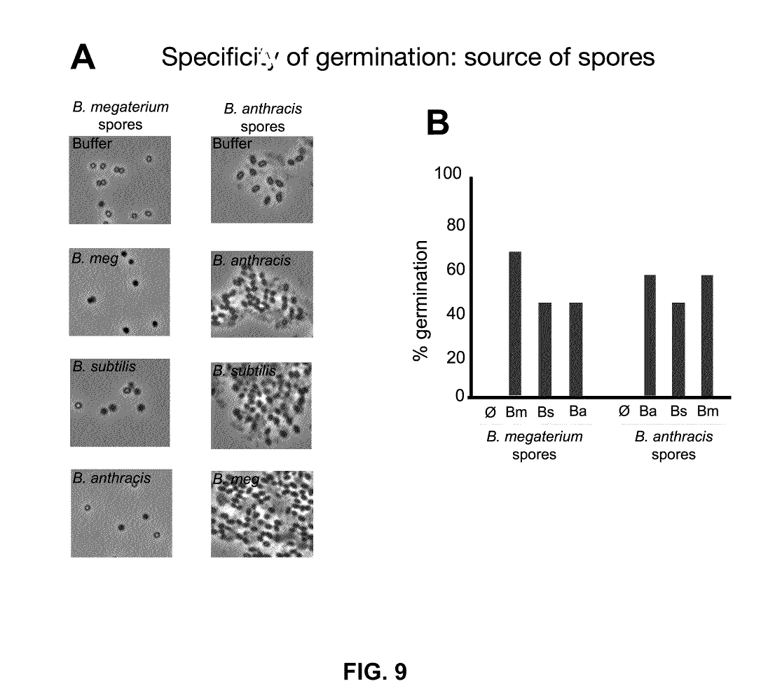

Spores of other spore-forming bacteria such as B. anthracis or B. megaterium also germinated in response to cell wall from other spore-formers (see e.g, FIG. 9).

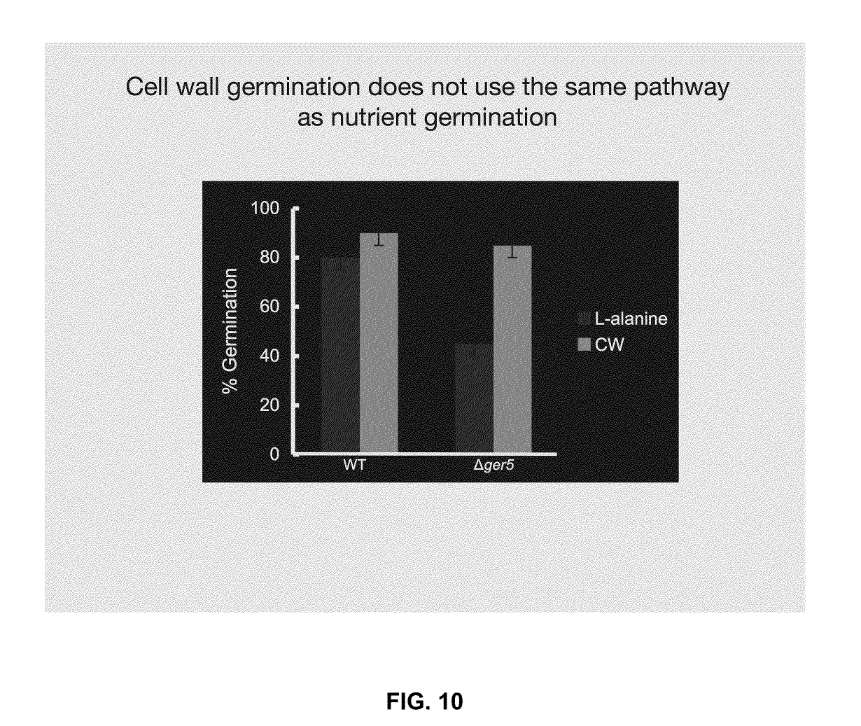

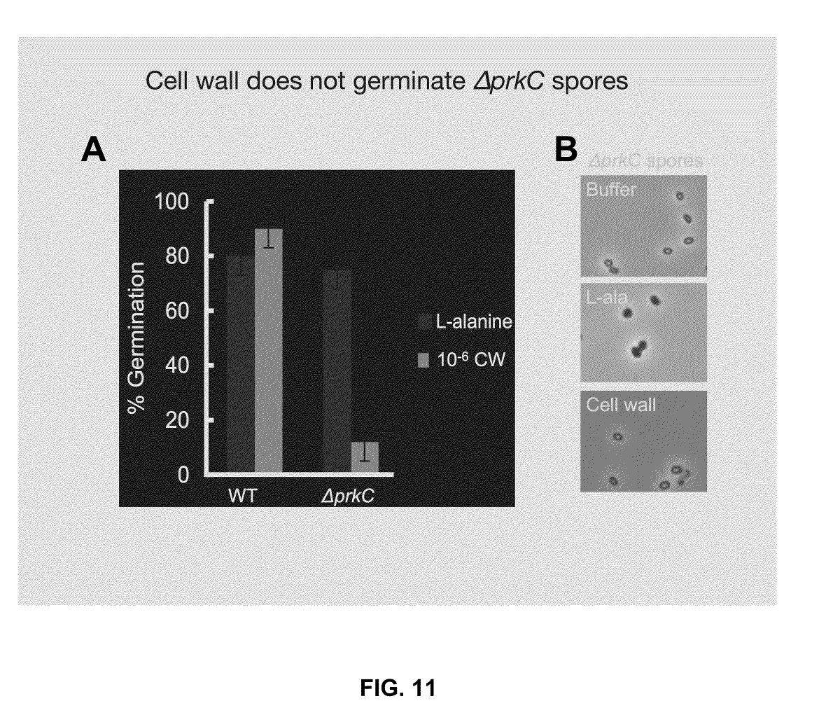

As shown in FIG. 10, cell wall-induced germination does not use the same molecular mechanism as nutrient germination since genetic deletion of all the receptors known to be essential for nutrient germination (Δger5) had no affect on spore germination in response to cell wall. In addition, D-alanine, which acts a competitive inhibitor of germination in response to L-alanine also did not block cell wall dependent germination. Additionally, spores derived from a strain lacking PrkC do not germinate in response to cell wall, although they still respond to alanine (see e.g, FIG. 11). This is also true for B. anthracis (data not shown).

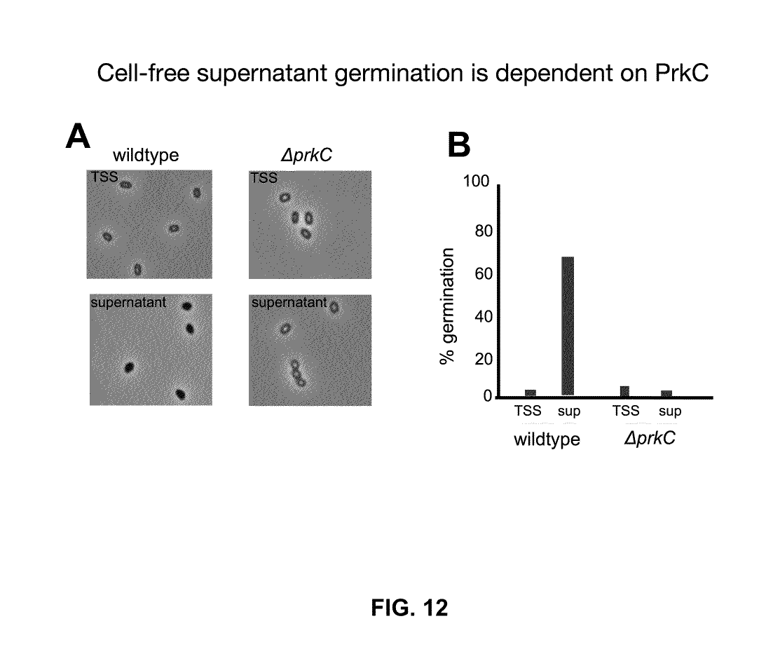

In addition to purified cell wall, supernatant from growing cells acts to induce germination (see e.g, FIG. 12). This suggests that cell wall released from a growing cell can act to induce germination in a neighboring spore.

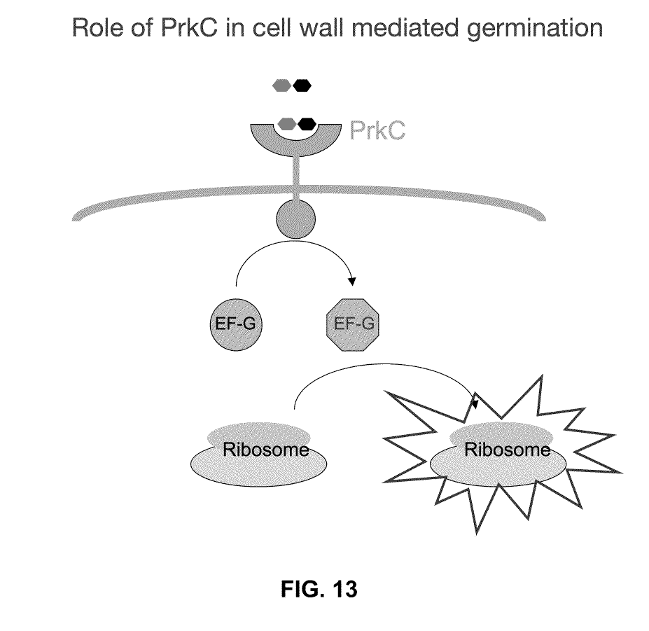

The only known downstream target of PrkC is the protein EF-G (elongation factor G) an essential G-protein that binds to the ribosome and stimulates its activity (see e.g, FIG. 13). Thus, binding of cell wall fragments to PrkC could lead to stimulation of its kinase activity, and phosphorylation of EF-G, which would then increase translation.

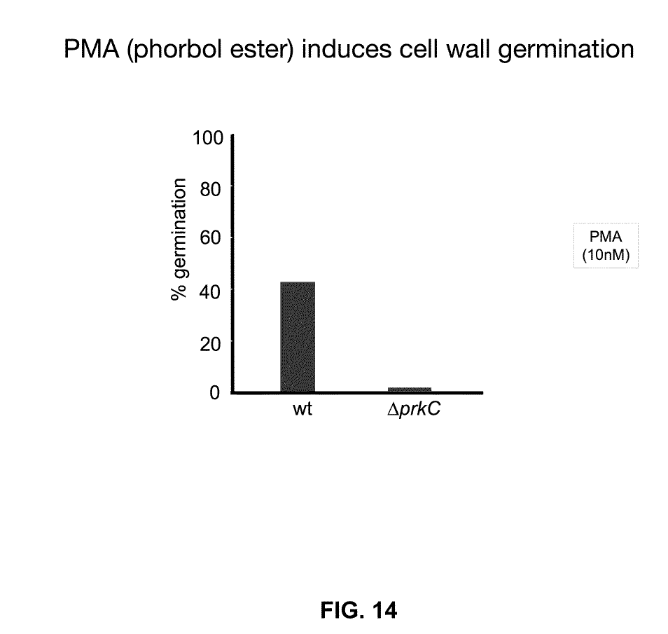

It was also determined that a kinase activator, the phorbol ester phorbol-12-myristate-13-acetate (PMA) chemically induces germination (see e.g, FIG. 14). The effect of the phorbol ester is dependent on the presence of PrkC, since spores of a strain lacking the prkC gene (ΔprkC) was not stimulated to germinate by PMA (see e.g, FIG. 14). This demonstrates the specificity of the PMA action.

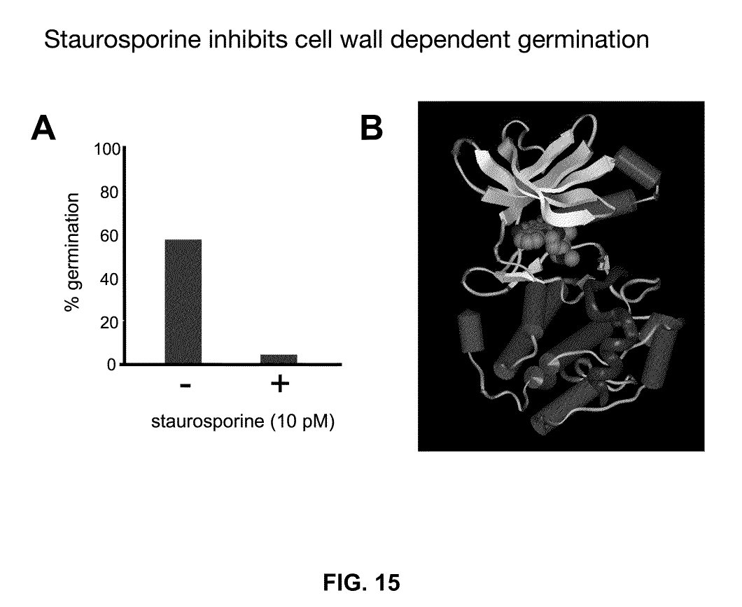

Further, the stimulation of spore germination by cell wall is inhibited with the small molecular kinase inhibitor staurosporine, a natural product of another soil bacterium, at pM concentrations (see e.g, FIG. 15). A model of the binding of a kinase by staurosporine is provided in FIG. 15B.

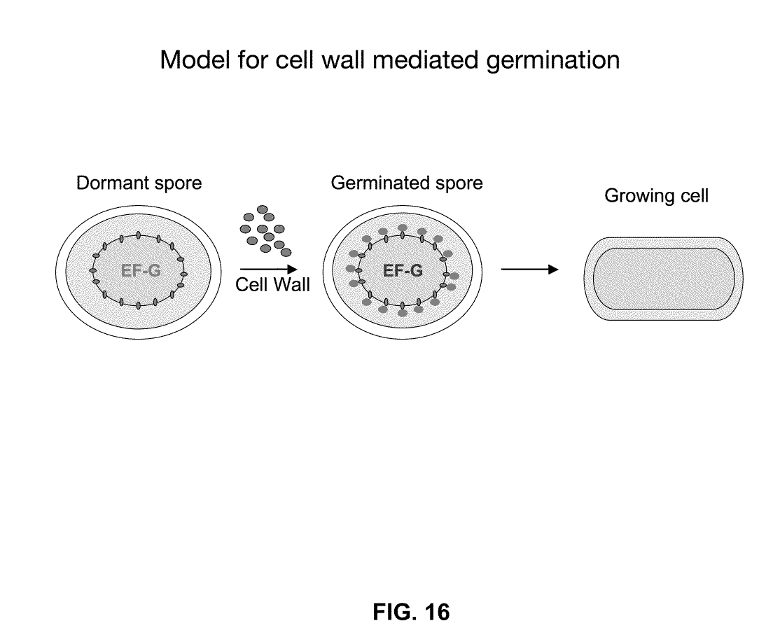

Thus, a model is provided where a dormant spore interacts with exogenously produced cell wall leading to a germinated spore where PrkC phosphorylates EF-G leading to increase in translation, and, ultimately to bacterial growth (see e.g, FIG. 16).

### Example 2

### Generation of Mtb Kinase-Expressing Bacillus

JDB2096: PB705 was transformed with pIMS50(pDR111-PknB) (SEQ ID NO: 1). The gene encoding pknB was amplified from Mtb Erdman genomic DNA using primers that included the B. subtilis prkC RBS followed by codons for FLAG tag after the start codon. The resulting PCR product was digested with NheI and SphI and the digested product was ligated to pDR111 digested with NheI and SphI.

pIMS41 (His₆-PrkC): Full length prkC was amplified from B. subtilis genomic DNA from strain PY79 using primers that included the native prkC RBS followed by six codons coding for histidine residues after the start codon. The resulting PCR product was digested with SpeI and SphI and the digested product was ligated to pDR111 digested with NheI and SphI.

pIMS40 (His₆-PASTA): Sequence corresponding to codons 357-648 (nt 1071-1944) of prkC was amplified from B. subtilis genomic DNA using primers that included six codons coding for histidine residues after the start codon. The resulting PCR product was digested with NcoI and XbaI and ligated to pBAD24 digested with NcoI and XbaI.

pIMS36 (His₆-Yycl): Sequence corresponding to codons 31-280 (nt 93-840) of yycl was amplified from B. subtilis genomic DNA using primers that included six codons coding for histidine residues after the start codon. The resulting PCR product was digested with NcoI and XbaI and ligated to pBAD24 digested with NcoI and XbaI.

### Example 3

### Generation of S. Aureus Kinase-Expressing Bacillus

JDB2017: PB705 was transformed with pIMS46(pDR111-Sa) (SEQ ID NO: 2). The gene encoding S_TKc was amplified from S. aureus NEWMAN genomic DNA using primers that included the B. subtilis prkC RBS followed by six codons coding for histidine residues after the start codon. The resulting PCR product was digested with NheI and SphI and the digested product was ligated to pDR111 digested with NheI and SphI.

pIMS46 (His₆-PrkC_{Sa}): The gene encoding S_TKc was amplified from S. aureus COL genomic DNA using primers that included the B. subtilis prkC RBS followed by six codons coding for histidine residues after the start codon. The resulting PCR product was digested with NheI and SphI and the digested product was ligated to pDR111 digested with NheI and SphI.

### Example 4

### Measurement of Germination by Loss of Heat Resistance

Spores were incubated at 10⁸ spores/ml in 50 μl reactions with germinant in germination buffer (10 mM Tris (pH 8), 1 mM glucose) for L-alanine or dH₂O for muropeptides for 60 min at 37° C. and then subjected to wet heat (80° C.) for 20 min. Heat-treated samples were diluted 10⁵-fold and 100 μL of the diluted samples were spread on LB-agar plates and following overnight incubation at 37° C., CFUs were determined. Loss of heat resistance as compared to that in the case of incubation with buffer (negative control) and 1 mM L-alanine (positive control) served as a marker for spore germination. Percent germination was expressed upon normalization using CFUs obtained with buffer as control which results in a failure to germinate spores (i.e., no change in CFUs before or after exposure to heat).

### Example 5

### Measurement of Germination by Fluorescence

Reactions were set up with 15 μL spores (10⁸ spores/ml final concentration) plus 135 μL germination mix (either non-germinant buffer or 1 μM Bryostatin (Calbiochem) final concentration or CFS) plus 5 μL Syto-9 dye (100 nM final concentration). Upon 5′ incubation at 37° C., fluorescence was read with excitation/emission of 485/530.

TABLE 1
Fluorescence Germination Results

| | Buffer | Bryostatin |
|---|---|---|
| Wild-type | 9057 | 14331 |
| ΔprKC (kinase mutant) | 4711 | 5208 |
| ΔprpC (phosphatase mutant) | 5083 | 13183 |
| ΔprpC, PrpC | 5300 | 5529 |
| (Phosphatase mutant overexpressing | | |
| phosphatase from an ectopic site) | | |

### Example 6

### Stimulation of Germination Makes Spores Sensitive to an Antibiotic

B. subtilis wild type spores were incubated with non-germinant buffer, muropeptide (GlcNAc-MurNAc tripeptide, 40 μM) (Anaspec), B. subtilis cell free supernatant, or bryostatin (Calbiochem) (1 μM), for 60 min at 37° C. prior to treatment with tetracycline (10 μg/ml for 60 min at 37° C.). Percent loss in plating efficiency was calculated relative to that observed in the absence of germinant.

TABLE 2
Sensitivity to antibiotic after stimulation of germination

| Germinant | % Loss in viable cells |
|---|---|
| None | 0 |
| Muropeptide | 72 ± 3 |
| Cell free supernatant | 45 ± 2 |
| Bryostatin | 32 ± 7 |

### Example 7

### Experimental Methods

General methods and bacterial strains. B. subtilis strains used in this study and relevant construction details are described in Example 2 and the Supplemental Data below. B. subtilis spores were prepared by growth to exhaustion in DSM medium, addition of lysozyme (1 mg/ml, 1 h, 37° C.) and SDS (2%) for 20 min at 37° C. Spores were washed 3× with dH₂O, resuspended in dH₂O and stored at 4° C. JDB1980, JDB2226, JDB2227 and JDB2017 spores carrying inducible copies of the PrkC_{Bs} and PrkC_{Sa} genes, respectively, were generated as above except that growth in DSM was in the presence of 1 mM IPTG.

Peptidoglycan Isolation. 100 ml cells grown in LB to an OD₆₀₀ ˜1.2 were collected by centrifugation, washed with 0.8% NaCl, resuspended in hot 4% SDS, boiled for 30 min and incubated at RT overnight. The suspension was then boiled for 10 min and the SDS-insoluble cell wall material was collected by centrifugation at 15 k for 15 min at RT. The pellet containing cell wall peptidoglycan was washed 4× with water and finally resuspended in 1 ml sterile water. Boiling twice with 4% SDS with an overnight incubation removes proteins and lipoteichoic acid molecules from the cell wall material (Girardin et al., 2003). The resuspended PG was digested with mutanolysin (10 μg/ml) overnight at 37° C. prior to inactivation of mutanolysin at 80° C. for 20 min and use of digested PG in germination assays. Peptidoglycan from B. anthracis Sterne, B. megaterium, B. sphaericus, L. innocua, E. coli, E. faecalis, S. aureus Newman, S. pyogenes, and L. casei were prepared similarly. Cell free supernatant was obtained from B. subtilis PY79 and E. coli DH5α cells grown in TSS medium, and from S. aureus Newman cells grown in Davis medium to an OD₆₀₀=1.2 by filtering (0.2 μm) the culture twice.

Purification of peptidoglycan fragments. B. subtilis vegetative peptidoglycan was purified, stripped of teichoic acids, and digested with mutanolysin (McPherson and Popham, 2003). Muropeptides were separated by HPLC using a phosphate buffer with a methanol gradient (Atrih et al., 1999) and individual muropeptides were collected upon elution from the HPLC column. The identities of purified muropeptides were verified using electrospray ionization mass spectrometry (Gilmore et al., 2004) and muropeptides were quantified relative to commercial purified amino acid standards using amino acid analysis.

Measurement of Germination. Spores were incubated at 10⁸ spores/ml in 50 μl reactions with germinant in germination buffer (10 mM Tris (pH 8), 1 mM glucose) for L-alanine or dH₂O for muropeptides for 60 min at 37° C. and then subjected to wet heat (80° C.) for 20 min. Heat treated samples were diluted 10⁵-fold and 100 μl of the diluted samples were spread on LB-agar plates and following overnight incubation at 37° C., CFUs were determined. Loss of heat resistance as compared to that in the case of incubation with buffer (negative control) and 1 mM L-alanine (positive control) served as a marker for spore germination. Percent germination was expressed upon normalization using CFUs obtained with buffer as control which results in a failure to germinate spores (i.e., no change in CFUs before or after exposure to heat).

Localization of FLAG-PrkC. Spores were decoated (as confirmed by loss of heat resistance), treated with TEP buffer in the presence of lysozyme, DNase and RNase for 5 min at 37° C., and cooled on ice for 20 min (Paidhungat and Setlow, 2001). Samples were sonicated (five 15 sec pulses) and debris was removed by centrifugation (14K, 5 min). The supernatant was centrifuged (100 k·g, 1 h) to isolate the soluble fraction and the membrane-containing pellet was resuspended in TEP buffer containing 1% Triton. Following separation of protein by SDS-PAGE (8%), the proteins were transferred onto nitrocellulose membrane prior to detection with anti-FLAG antibodies (Sigma) and ECL substrate (Amersham).

Peptidoglycan Binding. The C-terminal fragment of PrkC (His₆-PASTA_{Bs}) composed of residues 357-648 was purified using Ni²⁺ affinity chromatography with an E. coli strain carrying pIMS40 that overproduces His₆-PASTA_{Bs}. His₆-Yycl composed of residues 31-280 was purified using identical methodology using an E. coli strain carrying pIMS36. His₆-AcmA composed of residues 243-439 using an E. coli strain carrying pIMS42 and His₆-PASTA_{Sa} composed of residues 378-644 using an E. coli strain carrying pIMS44 were purified using identical methodology. 50 μg of proteins was separately incubated with purified B. subtilis or S. aureus peptidoglycan (˜5 mg) in 20 mM Tris-HCl, 50 mM MgCl₂, 500 mM NaCl for 30 min at 4° C. Centrifugation (10 min, 15 k) was employed to remove the supernatant (soluble fraction) and the pellet was washed twice, then resuspended it in 2% SDS and incubated at RT for 1 h. Bound fraction (insoluble fraction) was recovered by removing the insoluble pellet by centrifugation. Fractions consisting of unbound soluble protein and insoluble bound protein, and the wash were analyzed by SDS-PAGE. The gels were stained with Coomassie blue and the differences in the amounts of His₆-PASTA in the two fractions were determined by measurement of the appropriate bands using ImageJ (NIH).

Detection of phosphorylated EF-G. Spores were isolated from 100 ml cultures, decoated and treated either with non-germinant buffer or cell-free supernatant prior to treatment with TEP buffer (Lysozyme/DNase/RNase) and sonicated to remove debris. The resulting supernatant was subjected to ultracentrifugation at 100 k·g for 1 h. The soluble S100 fraction from each sample was subjected to immunoprecipitation with EF-G antibodies (kind gift of W. Wintermeyer) prebound to Protein A Dynabeads (Invitrogen) and immunoprecipitated proteins were separated by 6% SDS-PAGE followed by transfer of proteins onto nitrocellulose membranes. Immunoblotting was performed with either EF-G antibodies or phosphothreonine antibodies (Zymed, Invitrogen) to detect phosphorylated EF-G using ECL substrate (Amersham).

Supplemental Data.

Reagents. Bryostatin and staurosporine were obtained from Calbiochem and Sigma, respectively. Muramyl-dipeptide was obtained from Sigma and tripeptide (Ala-Glu-Dpm) was obtained from Anaspec.

General methods. B. anthracis Sterne spores were generated by growing cells for 4 days in modified G medium followed by repeated washing with dH₂O and storage at 4° C.

Antibiotic Sensitivity. B. subtilis wild type spores were incubated with non-germinant buffer, muropeptide (GlcNAc-MurNAc tripeptide, 40 μM), B. subtilis cell free supernatant, or bryostatin (1 μM), for 60 min at 37° C. prior to treatment with tetracycline (10 μg/ml for 60 min at 37° C.). Percent loss in plating efficiency was calculated relative to that observed in the absence of germinant

Plasmid Construction.

pIMS36 (His₆-Yycl): Sequence corresponding to codons 31-280 (nt 93-840) of yycl was amplified from B. subtilis genomic DNA using primers that included six codons coding for histidine residues after the start codon. The resulting PCR product was digested with NcoI and XbaI and ligated to pBAD24 digested with NcoI and XbaI.

pIMS40 (His₆-PASTA_{(Bs)}): Sequence corresponding to codons 357-648 (nt 1071-1944) of prkC was amplified from B. subtilis genomic DNA using primers that included six codons coding for histidine residues after the start codon. The resulting PCR product was digested with NcoI and XbaI and ligated to pBAD24 digested with NcoI and XbaI.

pIMS41 (His₆-PrkC): Full length prkC was amplified from B. subtilis PY79 genomic DNA using primers that included the native prkC RBS followed by six codons coding for histidine residues after the start codon. The resulting PCR product was digested with SpeI and SphI and the digested product was ligated to pDR111 digested with NheI and SphI.

pIMS42 (His₆-AcmA): Sequence corresponding to codons 243-439 of acmA was amplified from L. lactis genomic DNA (kind gift from M. Belfort) using primers that included six codons coding for histidine residues after the start codon. The resulting PCR product was digested with NcoI and XbaI and ligated to pBAD24 digested with NcoI and XbaI.

pIMS44(His₆-PASTA_{(Sa)}): Sequence corresponding to codons 378-644 of S_TPK was amplified from S. aureus NEWMAN genomic DNA using primers that included six codons coding for histidine residues after the start codon. The resulting PCR product was digested with NcoI and XbaI and ligated to pBAD24 digested with NcoI and XbaI.

pIMS46 (His₆-PrkC_{Sa}): The gene encoding S_TKc was amplified from S. aureus NEWMAN genomic DNA using primers that included the B. subtilis prkC RBS followed by six codons coding for histidine residues after the start codon. The resulting PCR product was digested with NheI and SphI and the digested product was ligated to pDR111 digested with NheI and SphI.

pIMS47 (FLAG-PrkC_{Bs}): Full length prkC was amplified from B. subtilis genomic DNA from strain PY79 using primers that included the native prkC RBS followed by codons coding for FLAG tag after the start codon. The resulting PCR product was digested with SpeI and SphI and the digested product was ligated to pDR111 digested with NheI and SphI.

pIMS48 (FLAG-PrkC_{Bs(K40A)}): pIMS47 was subjected to site-directed mutagenesis with primers to substitute lysine at position 40 with an alanine. PCR products resulting from the 5′ FLAG-prkC primer and (K40A) reverse primer as well as from K40A forward primer and 3′ prkC primer were gel-purified and used as templates for PCR-SOEing using 5′FLAG-prkC and 3′ prkC primers. The resulting PCR product was digested with SpeI and SphI and the digested product was ligated to pDR111 digested with NheI and SphI.

Strain construction.

See Table 4.

JDB1980 (ΔprkC amyE::P_{spac}-his6-prkC_{Bs}): PB705 was transformed with pIMS41, selecting for Spec^{R} and screening for amy-.

JDB2226 (ΔprkC amyE::P_{spac}-FLAG-prkC_{Bs}): PB705 was transformed with pIMS47, selecting for Spec^{R} and screening for amy-.

JDB2227 (ΔprkC amyE::P_{spac}-FLAG-prkC_{Bs(K40A)}: PB705 was transformed with pIMS48, selecting for Spec^{R} and screening for amy-.

JDB2017 (ΔprkC amyE::P_{spac}-his6-prkC_{Sa}): PB705 was transformed with pIMS46, selecting for spec^{R} and screening for amy-.

JDB1930 (B. anthracis ΔprkC): The temperature sensitive plasmid pKS1 (Shatalin and Neyfakh, 2005) was used to construct a deletion mutation (ΔprkC::aphA3). A Kan^{R} cassette was introduced into the bas3713 gene that had been amplified from B. anthracis Sterne 34F2 strain genomic DNA. This construct was then introduced into pKS1, and the resulting plasmid (pML280) was transformed into B. subtilis PY79. A midiprep of the plasmid amplified in B. subtilis was used to electroporate B. anthracis Sterne. This strain was grown at 37° C. without antibiotic and then selected for the integration of the pML280 plasmid into the B. anthracis chromosome using antibiotic selection (kanamycin, 10 μg/ml) followed by PCR screening for the insertion in the correct locus. After a cycle at a permissive temperature (30° C.) with antibiotic, the excision of the plasmid (loss of the erythromycin resistance) and the insertion of the antibiotic cassette in the prkC gene was selected using antibiotic selection and a PCR screen using flanking primers of the locus.

TABLE 4
Bacterial Strains

| Strain | Genotype | Source |
|---|---|---|
| PY79 | Wild type | Lab collection |
| EB1451 | hisA1 argC4 metC3 tagO::erm | (D'Elia et al., |
| | | 2006) |
| PB705 | trpC2 prkCΔ1 | (Gaidenko et al., |
| | | 2002) |
| FB85 | ΔgerΔ::spc ΔgerB::cat ΔgerK::erm | (Paidhungat and |
| | ΔyndDEF::tet ΔyfkQRT::neo | Setlow, 2000) |
| JDB1930 | B. anthracis Sterne ΔprkC | This study |
| JDB1980 | ΔprkCΔ1 amyE::P_{spac}-his₆-prkC | This study |
| JDB2017 | ΔprkCΔ1 amyE::P_{spac}-his₆-prkCₛₐ | This study |
| JDB2226 | ΔprkCΔ1 amyE::Pspac-FLAG-prkC_{Bs} | This study |
| JDB2227 | ΔprkCΔ1 amyE::Pspac-FLAGprkC_{Bs(K40A)} | This study |
| B. anthracis Sterne 34F2 | Wild type | Lab collection |
| B. megaterium MS021 | ΔbgaR/bgaM | Lab collection |
| C. acetobutylicum NCTC 619 | Wild type | ATCC #4259 |
| B. sphaericus 2362 | Wild type | Lab collection |
| L. innocua | Wild type | D. Portnoy |
| E. coli DH5α | hsdR17(r_{K}⁻m_{K}⁺) supE44 thī recA1 gyrA (Nal^{r}) | Lab collection |
| | relA1 D(laclZYA-argF)U169 deo^{R} | |
| | (F80ΔlacD(lacZ)M15) | |
| S. aureus Newman | Wild type | F. Lowy |
| E. faecalis OG1RF | Wild type | D. Garsin |
| S. pyogenes | Wild type | A. Ratner |
| L. casei | Wild type | A. Ratner |

### Example 8

### A Eukaryotic-Like Ser/Thr Kinase Signals Bacteria to Exit Dormancy in Response to Peptidoglycan Fragments

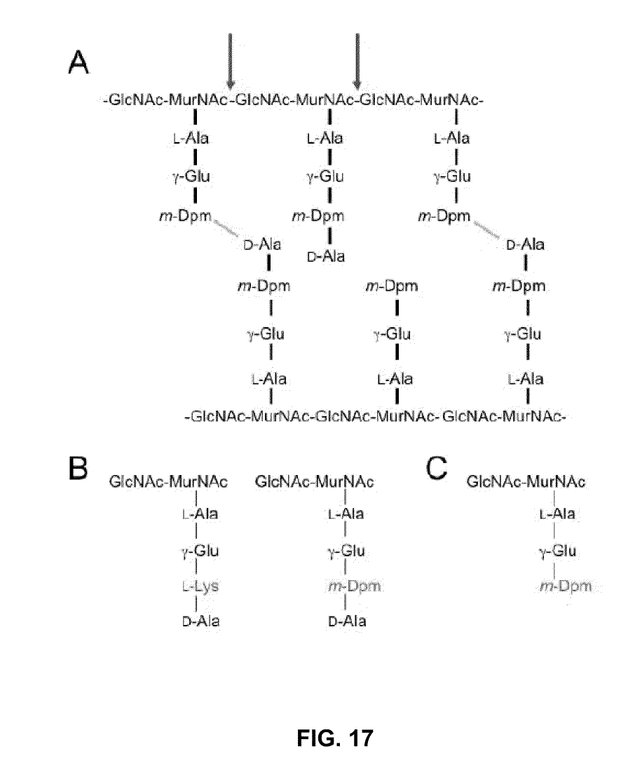

Bacterial shape and cellular resistance to cytoplasmic turgor pressure are determined by peptidoglycan, a polymer of repeated subunits of a N-acetylglucosamine (GlcNAc) and N-acetylmuramic acid (MurNAc) peptide monomer that surrounds the cytoplasmic membrane (FIG. 17A). Covalent interactions between the stem peptides arising from separate chains typically cross-link the GlcNAc-MurNAc polymers, although in some organisms this cross-bridge is composed of one or more amino acids. Most Gram-positive bacteria contain an L-Lysine residue at the third position of the stem peptide (FIG. 17B, left) whereas Gram-negative bacteria and most endospore-formers have an m-Dpm residue in this position (FIG. 17B, right).

Peptidoglycan fragments serve as signals in a range of host-microbe interactions including B. pertussis infection and V. fischeri-squid symbiosis (Cloud-Hansen et al., 2006). They also stimulate the innate immune response (Hasegawa et al., 2006) by binding to host proteins like Nod1 (Girardin et al., 2003). Peptidoglycan fragments are generated by growing cells as peptidoglycan hydrolases and amidases partially digest the mature peptidoglycan to allow insertion of additional peptidoglycan monomers (Doyle et al., 1988). While Gram-negative bacteria can efficiently recycle the resulting muropeptides, the lack of a similar recycling system in Gram-positive bacteria results in the release of large quantities of peptidoglycan fragments into the extracellular milieu by growing cells (Doyle et al., 1988; Mauck et al., 1971).

Dormant bacteria must monitor nutrient availability so that they can reinitiate metabolism when conditions become favorable. This could be accomplished by determining changes in the levels of individual nutrients. Alternatively, the growth of other bacteria in the environment would also indicate the presence of favorable conditions. Since growing bacteria release muropeptides into the environment, these molecules could serve as an intercellular growth signal to dormant bacteria.

Some Gram-positive species produce dormant spores under conditions of nutritional limitation. These cells are resistant to harsh environmental conditions and can survive in a dormant state for years (Nicholson et al., 2000). Spores exit from dormancy via the process of germination that is triggered by specific molecules known as germinants. Most spore-forming bacteria encode several germination receptors; for example, the B. subtilis GerAA/AB/AC proteins are necessary for germination in response to L-alanine. GerAA and GerAB are integral membrane proteins and GerAC is a putative lipoprotein. GerAA and GerAC, and GerBA, a GerAA homolog, are located in the inner membrane of the spore (Hudson, 2001; Paidhungat and Setlow, 2001) where they are positioned to detect germinants that can pass through the outer layers of the spore. The precise chemical nature of germinants varies according to the species, and although they are typically nutrients, these molecules are not metabolized. The amino acid L-alanine or a mixture of asparagine, glucose, fructose and potassium ions germinates B. subtilis spores, whereas L-proline germinates B. megaterium spores and purine ribonucleosides and amino acids act as co-germinants for B. anthracis spores (Setlow, 2003).

High concentrations of nutrient germinants would be consistent with the ability of the environment to support the growth of germinated spores. However, a more integrated determination of this ability is the growth of other microbes in the environment and this growth would be indicated by the presence of released muropeptides. How might dormant spores recognize these muropeptides? One protein sequence hypothesized to bind peptidoglycan is the PASTA (Penicillin and Ser/Thr kinase Associated) repeat found in the extracellular domain of membrane-associated Ser/Thr kinases as well as in some proteins that catalyze the transpeptidation reaction in cell wall synthesis. The PASTA domain is a small (˜55 aa) globular fold consisting of 3 beta-sheets and an alpha-helix, with a loop region of variable length between the first and second beta-strands (Yeats et al., 2002). While the presence of PASTA domains in proteins that interact with peptidoglycan suggests that these domains may mediate this interaction, the binding of PASTA domains to peptidoglycan has not been demonstrated.

The cytoplasmic kinase domain of M. tuberculosis PknB, the essential PASTA-domain containing Ser/Thr kinase, is structurally homologous to eukaryotic Ser/Thr kinases (Young et al., 2003). Consistent with this homology, PknB phosphorylates several proteins, including a transcriptional activator (Sharma et al., 2006) and a cell division protein (Dasgupta et al., 2006). The closely related B. subtilis PASTA-domain-containing Ser/Thr kinase, PrkC, phosphorylates elongation factor G (EF-G) both in vivo and in vitro. EF-G is an essential ribosomal GTPase involved in mRNA and tRNA translocation (Gaidenko et al., 2002) and although the activity of its eukaryotic homolog, eEF-2 (Ryazanov et al., 1988) is regulated by phosphorylation, similar data are not available for EF-G. While PrkC is not essential, ΔprkC strains have decreased viability (˜1 log) following incubation in stationary phase for >24 h (Gaidenko et al., 2002) and are moderately defective for sporulation (Madec et al., 2002).

As shown here, muropeptides, purified peptidoglycan or supernatants derived from cultures of growing cells are potent germinants of dormant B. subtilis spores. Diverse bacteria can serve as the source of these molecules, but the identity of a single amino acid residue in the peptidoglycan stem peptide determines its ability to induce germination. PrkC is necessary for this germination response and several small molecules known to affect the activity of related eukaryotic kinases either stimulate or inhibit germination.

Methods are according to Example 7 unless indicated otherwise.

Results showed that cell-free supernatant causes B. subtilis spores to germinate. Dormant bacteria must continuously monitor conditions so that they can reinitiate metabolism when conditions become favorable. The growth of other bacteria in the local environment would reflect such changes and this growth could be assayed by detecting released metabolic byproducts. These molecules would then serve as a signal for dormant cells that conditions conducive to growth are present. For example, dormant spores would be germinated by supernatants derived from growing bacterial cultures.

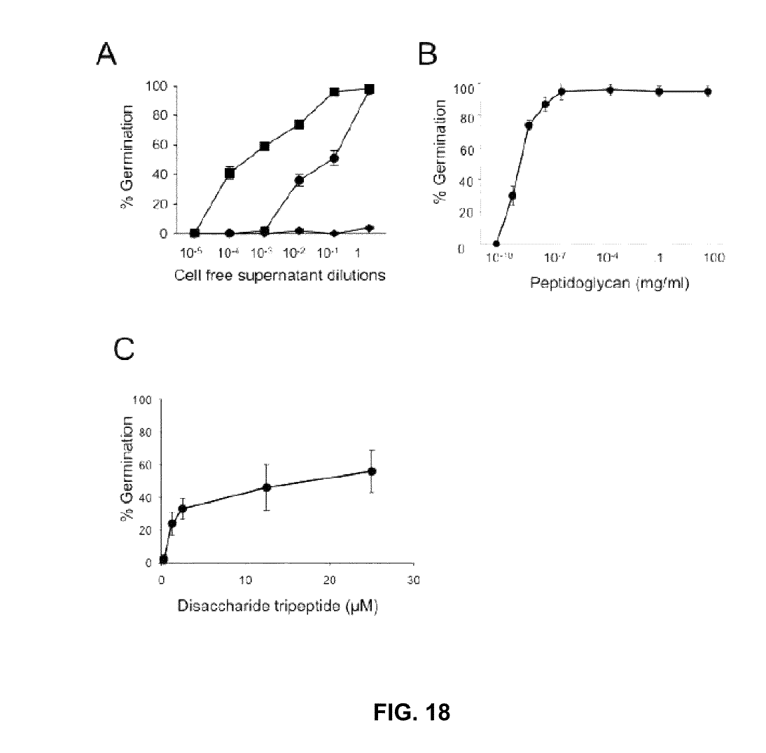

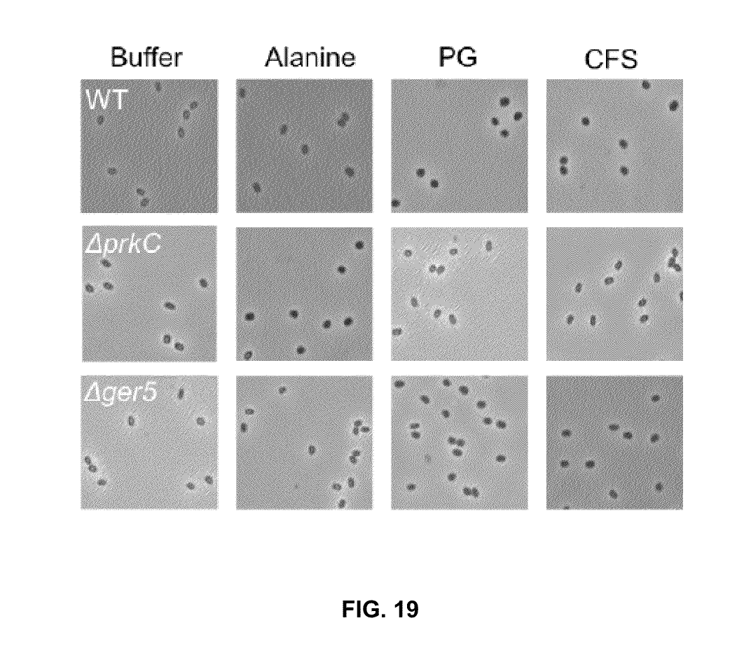

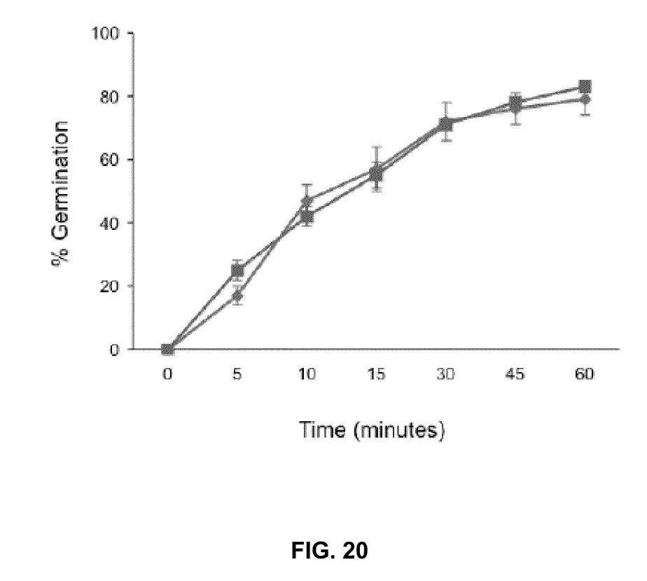

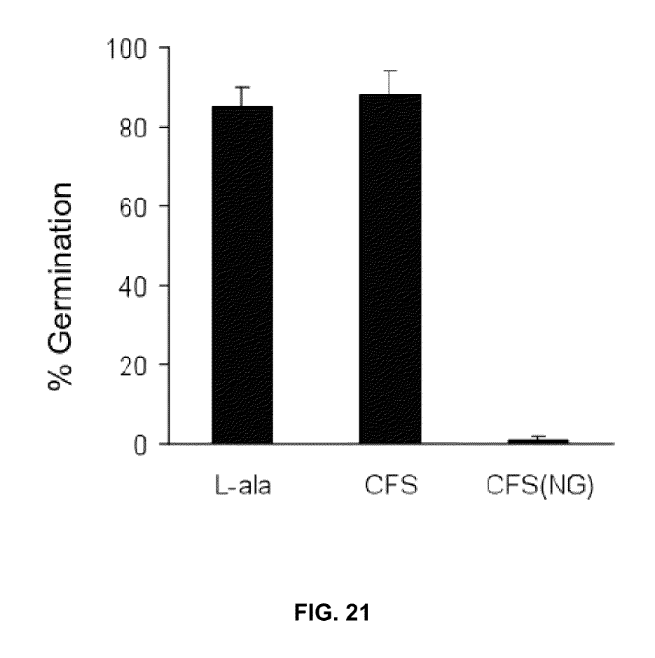

This possibility was tested by growing B. subtilis and removing the cells from the supernatant by repeated filtration. Germination was assayed by measuring loss of heat resistance because dormant, but not germinated, spores are resistant to wet heat. Incubation of cell-free supernatants from B. subtilis cultures induced germination of dormant spores (FIG. 18A, squares). This germination caused phase-bright spores to become phase-dark (FIG. 19) and occurred with similar kinetics as seen with nutrient germination (FIG. 20). However, cell-free supernatants from the Gram-positive bacterium S. aureus did not induce germination indicating that the stimulatory component was not generated by this species (FIG. 18A, diamonds). Supernatants from E. coli cultures were also effective, albeit with decreased potency (FIG. 18A, circles). The reduced effectiveness of E. coli supernatants likely results from the presence of the outer membrane that acts as a permeability barrier for hydrophilic compounds in the periplasm (Beveridge, 1999) and therefore inhibits the release of molecules from the cell. However, the ability of cell-free supernatants derived from a Gram-positive and a Gram-negative species to induce germination suggests that the molecule(s) responsible are likely to be released by a phylogenetically broad range of bacteria. Finally, since supernatants isolated from cells transferred to non-growth medium failed to efficiently germinate spores (FIG. 21), these molecules are likely to be produced only by growing cells.

### Example 9

### Peptidoglycan Causes B. Subtilis Spores to Germinate

The increased spore germination induced by B. subtilis cell free supernatant as compared to E. coli is consistent with the larger release of peptidoglycan fragments by Gram-positive as compared to Gram-negative bacteria (Goodell and Schwarz, 1985). Thus, peptidoglycan fragments may act as a spore germinant. To examine this possibility, peptidoglycan was purified from growing B. subtilis cells and digested into muropeptides with mutanolysin, an enzyme that hydrolyzes the β-1,4 bond between the MurNAc and GlcNAc sugars (arrow, FIG. 17A).

Methods are according to Example 7 unless indicated otherwise.

Concentrations of peptidoglycan as low as ˜0.1 pg/ml induced germination (FIG. 18B), indicating that spores detected one or more peptidoglycan fragment. This amount of peptidoglycan corresponds to <1 B. subtilis cell based on our isolation of ˜100 mg peptidoglycan from a 100 ml B. subtilis culture grown to O.D. of 1.2. B. subtilis peptidoglycan also germinated spores generated by other Bacilli including B. anthracis and B. megaterium (data not shown), indicating that the peptidoglycan germination signal is not genus specific.

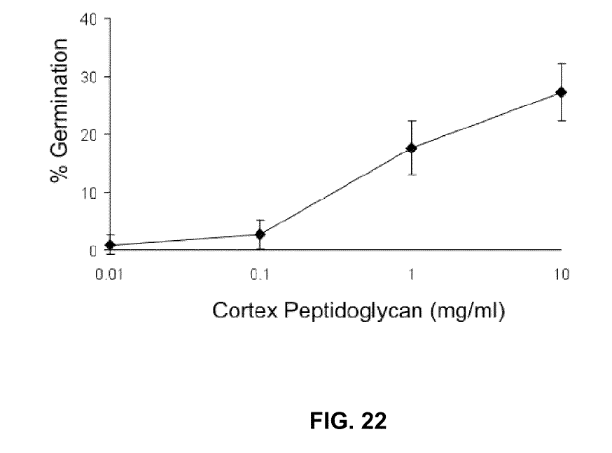

Bacterial peptidoglycan is often covalently associated with proteins and the anionic polymer teichoic acid. However, treatment of peptidoglycan with the proteases pronase and trypsin did not reduce its ability to act as a germinant (data not shown). In addition, peptidoglycan generated from a B. subtilis tagO mutant that is unable to synthesize teichoic acids (D'Elia et al., 2006) is similarly active as a germinant (data not shown). Thus, peptidoglycan fragments themselves are most likely to be the spore germinant. Further, peptidoglycan isolated from the spore cortex fails to efficiently function as a spore germinant (FIG. 22), indicating that only peptidoglycan released by or isolated from vegetative cells functions as a germinant.

### Example 10

### Muropeptides Act as Spore Germinants

The ability of both purified mutanolysin-digested peptidoglycan and cell-free supernatant to germinate spores suggested that muropeptides present in both preparations was responsible. This possibility was examined by separating mutanolysin-digested B. subtilis peptidoglycan into its muropeptide constituents by high-performance liquid chromatography.

Methods are according to Example 7 unless indicated otherwise.

Incubation of disaccharide tripeptides with dormant B. subtilis spores at concentrations as low as 1 μM (FIG. 18C) led to germination. In addition, disaccharide tetrapeptides were equivalently effective as germinants (data not shown). However, the concentrations of purified disaccharide tripeptides required for a germination response (μM) are higher than the concentration of muropeptides resulting from directly digesting peptidoglycan with mutanolysin (pM). One likely explanation for this difference is the substitution of muramic acid to muramitol due to a reduction step before HPLC purification. Further, both muramyl dipeptide (1 mM, data not shown) and an Ala-D-γ-Glu-Dpm tripeptide (500 μM, data not shown) failed to induce germination, suggesting that both the disaccharide and the third residue in the stem peptide play an important role. Thus, a disaccharide tripeptide appears to be the minimal chemical unit sufficient to germinate spores. Interestingly, a similar requirement is observed with a human peptidoglycan recognition protein heterodimer that binds tracheal cytotoxin where the disaccharide bridges the two proteins (Chang et al., 2006; Lim et al., 2006).

### Example 11

### Muropeptide Specificity

The ability of both supernatants derived from cultures of growing B. subtilis and E. coli, but not S. aureus, to induce germination (FIG. 18A) could be the result of the presence of a m-Dpm (meso-diaminopimelic acid) residue in the third position of their stem peptides (FIG. 17B, right). S. aureus, like most Gram-positive bacteria, has an L-Lys at that position (Schleifer and Kandler, 1972), so the identity (m-Dpm vs. L-Lys) of the third residue in the stem peptide could play an important role in recognition of peptidoglycan by spores. This possibility was examined by purifying peptidoglycan from a number of Gram-positive species that contain different amino acids at the third position of the stem peptide and assaying their ability to induce germination.

Methods are according to Example 7 unless indicated otherwise.

Consistent with the prediction, only peptidoglycan containing m-Dpm at the third position acted as a strong germinant (Table 3). Peptidoglycan derived from the spore-former Bacillus sphaericus that, in contrast to all other Bacilli contains an L-Lys at this position (Hungerer and Tipper, 1969), did not strongly induce germination, highlighting the importance of this residue. Both the mammalian Nod1 protein selectively binds peptidoglycan fragments containing m-Dpm (Girardin et al., 2003) and the human peptidoglycan recognition protein heterodimer binds tracheal cytotoxin where the m-Dpm residue is the primary specificity determinant (Chang et al., 2006; Lim et al., 2006). Thus, the identity of the amino acid in the third position of the stem peptide is critical for the recognition of peptidoglycan by phylogenetically diverse proteins.

TABLE 3
Role of third residue of stem peptide in germination

| Species | Peptide | Germination |
|---|---|---|
| Bacillus subtilis | m-Dpm | +(85% ± 6) |
| Bacillus anthracis | m-Dpm | +(65% ± 8) |
| Bacillus megaterium | m-Dpm | +(57% ± 4) |
| Bacillus sphaericus | L-Lys | −(6% ± 5) |
| Clostridium acetobutylicum | m-Dpm | +(72% ± 8) |
| Listeria monocytogenes | m-Dpm | +(67% ± 3) |
| Streptomyces coelicolor | L,L-Dpm | +(77% ± 3) |
| Enterococcus faecalis | L-Lys | −(10% ± 4) |
| Staphylococcus aureus | L-Lys | −(5% ± 4) |
| Streptococcus pyogenes | L-Lys | −(8% ± 6) |
| Lactobacillus lactis | L-Lys | −(3% ± 2) |

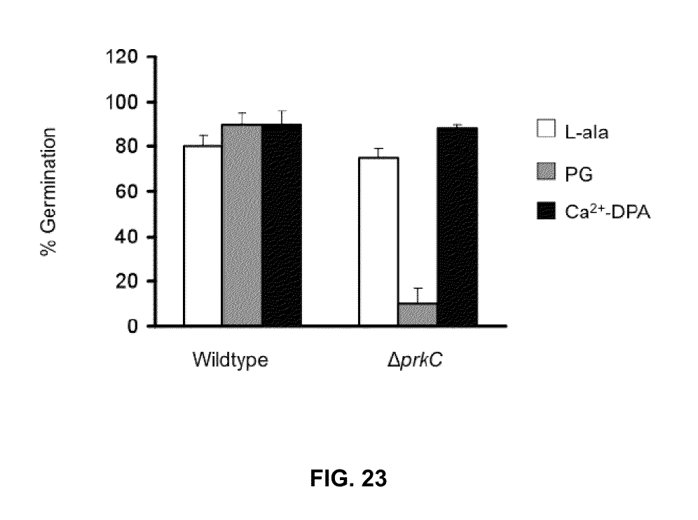

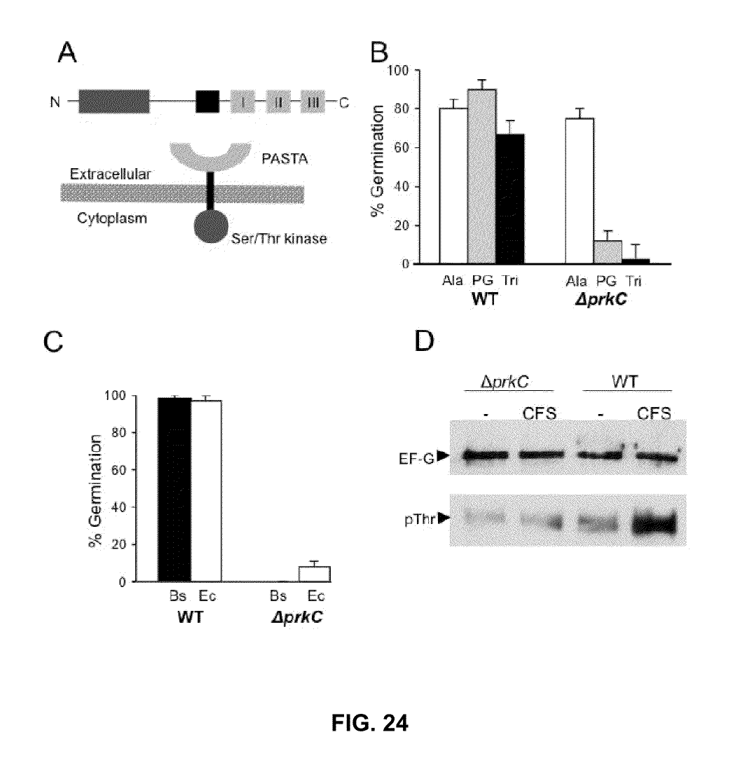

Muropeptides are recognized by a novel germination pathway. Nutrient germinants are detected by germination receptors located in the spore membrane. Since peptidoglycan fragments still germinated spores lacking all five previously identified germination receptors (Paidhungat and Setlow, 2000), these receptors were not involved in this response (FIG. 19). Therefore, to identify the relevant receptor for peptidoglycan fragments during germination, bacterial membrane proteins known or hypothesized to bind peptidoglycan were examined. Diverse bacteria including all known spore-forming bacteria have at least one eukaryotic-like Ser/Thr membrane kinase containing multiple PASTA repeats in their extracellular domains (FIG. 24A) that have been hypothesized to recognize the peptidoglycan stem peptide (Jones and Dyson, 2006; Yeats et al., 2002). It was therefore asked whether the B. subtilis member of this family, PrkC_{Bs}, is involved in peptidoglycan-dependent spore germination. Mutant spores lacking PrkC_{Bs} (ΔprkC) failed to germinate in the presence of peptidoglycan fragments or purified disaccharide tri-peptides (FIG. 24B) and tetra-peptides (data not shown). Thus, PrkC_{Bs} is required for the germination response of spores exposed to peptidoglycan. ΔprkC spores still responded to the nutrient germinant L-alanine (FIG. 24B), and to the chemical germinant Ca²⁺-dipicolinic acid (FIG. 23), indicating that the spores were still capable of germinating and that PrkC_{Bs} is not involved in nutrient or chemical germination.

Since growing cells release peptidoglycan fragments into the extracellular milieu, germination by cell-free supernatant should also require PrkC_{Bs}. In support of this hypothesis, incubation of ΔprkC_{Bs} spores with cell-free supernatant derived from either B. subtilis or E. coli cultures (FIG. 24C) did not result in germination. Although the identity of the component(s) in the supernatants necessary for germination is not known, the requirement for PrkC_{Bs} for germination in response to muropeptides suggests that these are likely to be the active molecules

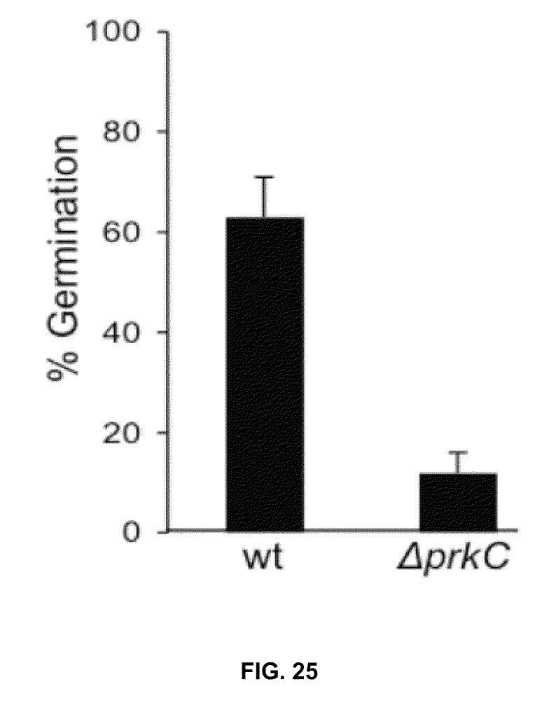

Finally, the requirement for PrkC was tested in another spore-former by constructing a deletion of the B. anthracis prkC homolog. Spores carrying this mutation were similarly blocked in the germination response to peptidoglycan (FIG. 25). Thus, the role of PrkC in germination is conserved in at least two spore-forming bacterial species.

### Example 12

### PrkC Phosphorylates EF-G During Germination

During vegetative growth of B. subtilis, phosphorylation of EF-G, an essential ribosomal GTPase, is reduced in a strain lacking PrkC. In addition, purified kinase domain of PrkC phosphorylates EF-G in vitro on at least one threonine (Gaidenko et al., 2002). Therefore, it was asked whether EF-G phosphorylation also occurs during PrkC-dependent germination.

Methods are according to Example 7 unless indicated otherwise.

Lysates were generated from wild-type and ΔprkC spores after incubation with cell free supernatant for 60 min to stimulate germination and immunoprecipitated EF-G using polyclonal antibodies raised against E. coli EF-G (kind gift of W. Wintermeyer). When these immunoprecipitated fractions were probed with an anti-phosphothreonine antibody (Zymed), it was observed that EF-G (as identified by probing the same fractions with the α-EF-G) phosphorylation increased following exposure to cell free supernatant (FIG. 24D). In contrast, no change in phosphorylation was observed in spores lacking PrkC.

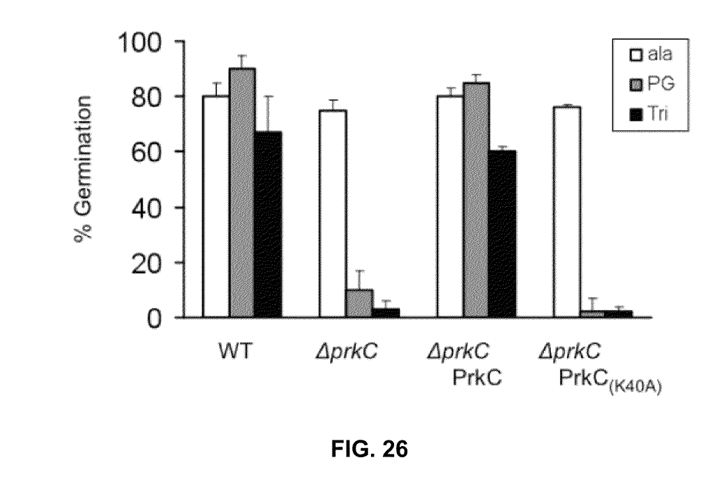

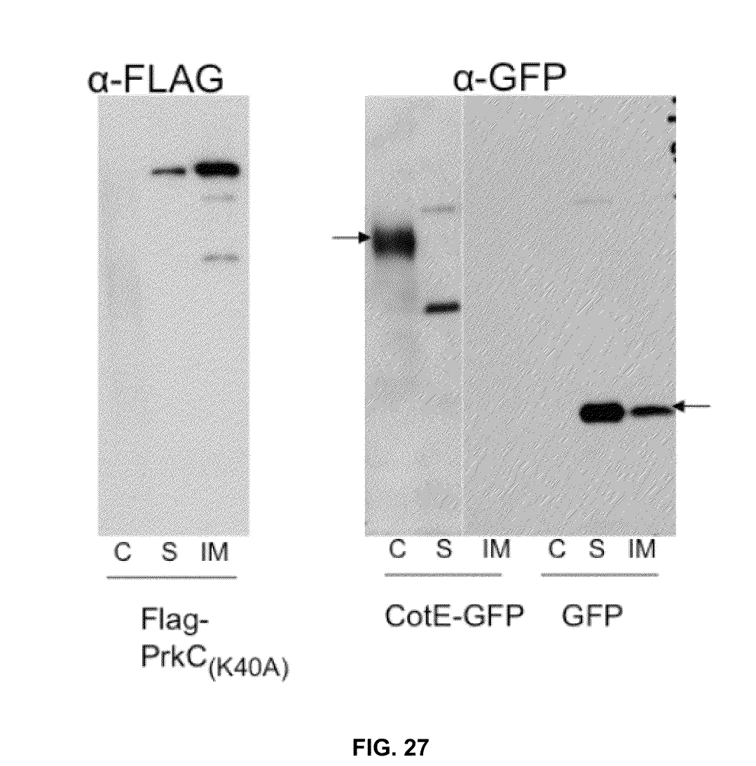

As a confirmation of the kinase activity of PrkC during germination, a FLAG-tagged point mutant (K40A) of PrkC was generated, since that residue was identified as necessary for PrkC phosphorylation (Madec et al., 2002). Consistent with the expected effect of this mutation, this mutant PrkC did not support germination in response to PG (FIG. 26) even though it was expressed and localized properly to the spore inner membrane (FIG. 27), whereas a FLAG-tagged version of the wild-type protein did complement a ΔprkC mutation. Thus, PrkC appears to phosphorylate EF-G during germination and this modification is likely necessary for germination in response to PG.

### Example 13

### PrkC Localizes to the Spore Inner Membrane

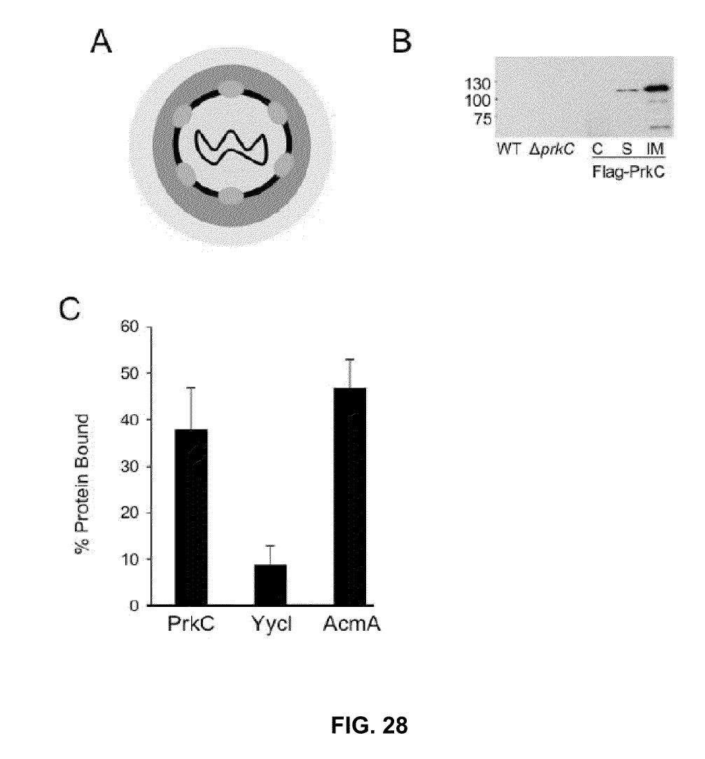

The inability of ΔprkC_{Bs} spores to germinate in response to muropeptides suggested that PrkC_{Bs} is located either on the spore surface or in the spore interior. The presence of a hydrophobic stretch between the cytoplasmic kinase and extracellular PASTA domains as well as the association of PrkC_{Bs} with the cytoplasmic membrane in vegetative cells (Madec et al., 2002) suggests that it is associated with the spore membrane, located below the spore coat (FIG. 28A). The critical hypothesis was tested that PrkC_{Bs} is membrane-associated in the spore and therefore strategically positioned to sense extracellular peptidoglycan by performing subcellular fractionation of an epitope-tagged PrkC protein.

Methods are according to Example 7 unless indicated otherwise.

Upon removal of the spore coat and outer membrane, it was observed that a FLAG-PrkC_{Bs} fusion protein, which complements a ΔprkC mutation for peptidoglycan-dependent germination (FIG. 26), was found in the inner membrane fraction of the spore (FIG. 28B) similar to proteins involved in nutrient germination (Hudson, 2001; Paidhungat and Setlow, 2001). These decoated spores still responded to PG as a germinant (data not shown). Spores expressing either free GFP under control of a forespore-specific promoter or a fusion of GFP to a coat protein exhibited expected patterns of fractionation (FIG. 27).

Since molecules>2-8 kDa are unable to cross the spore coat (Driks, 1999), peptidoglycan fragments that interact with PrkC proteins located in the spore inner membrane below the coat (FIG. 28) must not exceed this size. The observed ability of disaccharide tri- and tetra-peptide fragments (868.9 Da and 940.0 Da, respectively) to germinate spores is consistent with this requirement.

### Example 14

### Binding of Peptidoglycan by PrkC

The presence of the hypothesized peptidoglycan-binding PASTA repeats in the PrkC extracellular domain suggested that PrkC functions by binding peptidoglycan. This possibility was tested by expressing and purifying a His-tagged protein (His₆-PASTA_{Bs}) consisting of the entire extracellular domain of PrkC that contains three PASTA repeats.

Methods are according to Example 7 unless indicated otherwise.

Following previous characterization of bacterial proteins that bind peptidoglycan (Eckert et al., 2006; Steen et al., 2003), His₆-PASTA_{Bs} was incubated with purified B. subtilis peptidoglycan and the mixture centrifuged. In this assay, bound proteins pellet with the insoluble peptidoglycan molecules and unbound proteins remain in the supernatant. Under these conditions, His₆-PASTA_{Bs} remained soluble in the absence of added peptidoglycan (data not shown). The fractions were analyzed by SDS-PAGE and the differences in the protein amounts as revealed by Coomassie staining were quantified (FIG. 28C). Approximately 40% of the total protein was associated with the insoluble fraction, indicating that a substantial fraction of His₆-PASTA_{Bs} bound to peptidoglycan under the assay condition. As a control, the His-tagged extracellular domain of Yycl, a membrane associated histidine kinase from B. subtilis (Santelli et al., 2007), was expressed and purified. Consistent with its lack of PASTA domains, only ˜5% of the total protein was found in the insoluble fraction after incubation of this fragment with purified B. subtilis peptidoglycan. As a second control His₆-AcmA, a L. lactis protein that binds peptidoglycan, was examined in the assay and, like His₆-PASTA_{Bs}, approximately 40-45% protein remained associated to PG (FIG. 28C). Thus, the PASTA containing extracellular C-terminal domain of PrkC_{Bs} binds peptidoglycan, consistent with the model that PrkC_{Bs} directly binds to muropeptides during germination.

### Example 15

### Specificity of PrkC

Peptidoglycan containing an L-Lys at the third position of the stem peptide does not germinate B. subtilis spores, whereas peptidoglycan containing an m-Dpm at this position does act as a germinant (Table 3). Since PrkC is necessary for this germination and the PrkC extracellular domain binds peptidoglycan (FIG. 28C), this specificity may originate in PrkC. Thus, a PrkC homolog from a bacterium containing an L-Lys residue should respond to L-Lys containing peptidoglycan. This possibility was tested by substituting the PrkC homolog from the L-Lys containing species S. aureus (PrkC_{Sa}) for PrkC_{Bs} and determining whether spores expressing this heterologous protein germinated in response to L-Lys containing peptidoglycan.

Methods are according to Example 7 unless indicated otherwise.

The gene encoding PrkC_{Sa} was amplified from the S. aureus chromosome and placed under inducible control in the chromosome of a B. subtilis ΔprkC_{Bs} strain.

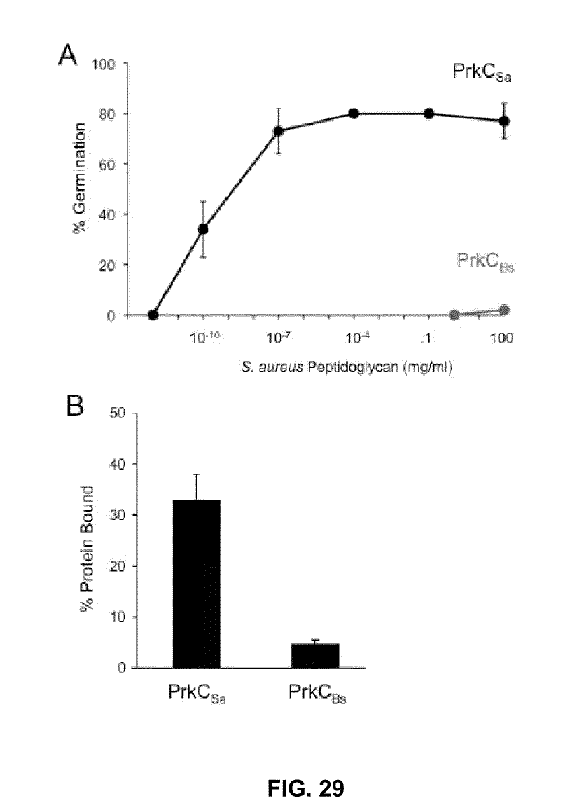

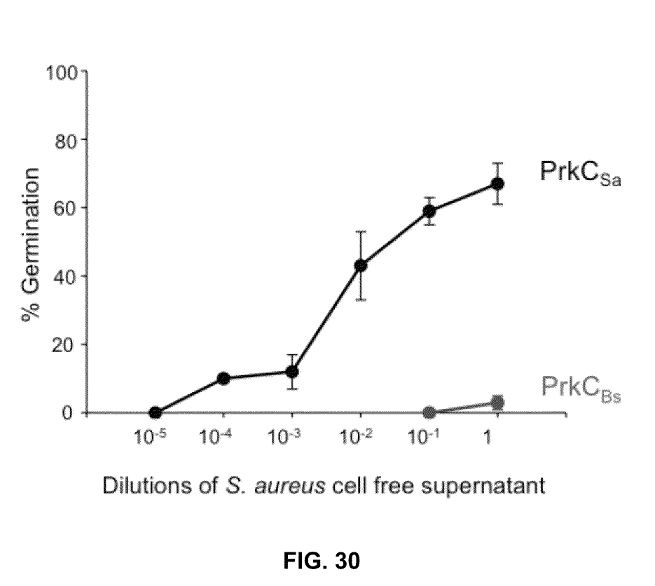

Transgenic PrkC_{Sa} expressing spores germinated in response to L-Lys containing S. aureus peptidoglycan (FIG. 29A, black) whereas wild type PrkC_{Bs} expressing spores did not germinate (FIG. 29A, red). Thus, the source of PrkC determined its ability to respond to L-lys containing peptidoglycan since PrkC_{Bs} responds to B. subtilis peptidoglycan (FIG. 24B). In addition, spores expressing PrkC_{Sa} germinated in response to B. subtilis peptidoglycan (data not shown), indicating that PrkC_{Sa} responds to both L-Lys and m-Dpm containing peptidoglycan. As a further test of this change in specificity, PrkCₛₐ expressing spores was incubated with S. aureus cell-free supernatant that does not germinate wild type B. subtilis spores. Consistent with the previous observations regarding germination in response to S. aureus peptidoglycan, S. aureus cell-free supernatant germinated PrkCₛₐ expressing spores (FIG. 30). Thus, L-Lys containing peptidoglycan can act as a germinant when the Ser/Thr PASTA containing kinase is changed.

Since the extracellular domain of PrkC_{Bs} binds to PG (FIG. 28C), it was examined whether the ability of S. aureus PG to act as a germinant of PrkC_{Sa} expressing spores was due to the ability of the extracellular domain of PrkC_{Sa} to bind S. aureus PG. In support of this interpretation, His₆-PASTA_{Sa} bound S. aureus PG much better than His₆-PASTA_{Bs} (FIG. 29B). Thus, the ability of PrkC_{Sa} expressing spores to germinate in response to S. aureus PG is at least in part due to the ability of these spores to bind to S. aureus PG.

### Example 16

### Regulation of Germination by Small Molecule Kinase Modulators

The cytoplasmic domain of the PrkC_{Bs} homolog, M. tuberculosis PknB, is structurally homologous to the catalytic domains of eukaryotic Ser/Thr kinases (Young et al., 2003). This similarity suggests that small molecules known to modulate the activity of these eukaryotic kinases might also modulate PrkC homologs. One of these molecules, bryostatin, a natural product synthesized by a marine bacterium, potently activates eukaryotic intracellular Ser/Thr kinases through direct binding to the phorbol ester binding site (Hale et al., 2002). It was examined whether bryostatin activated PrkC by incubating wild type B. subtilis spores with a range of bryostatin concentrations.

Methods are according to Example 7 unless indicated otherwise.

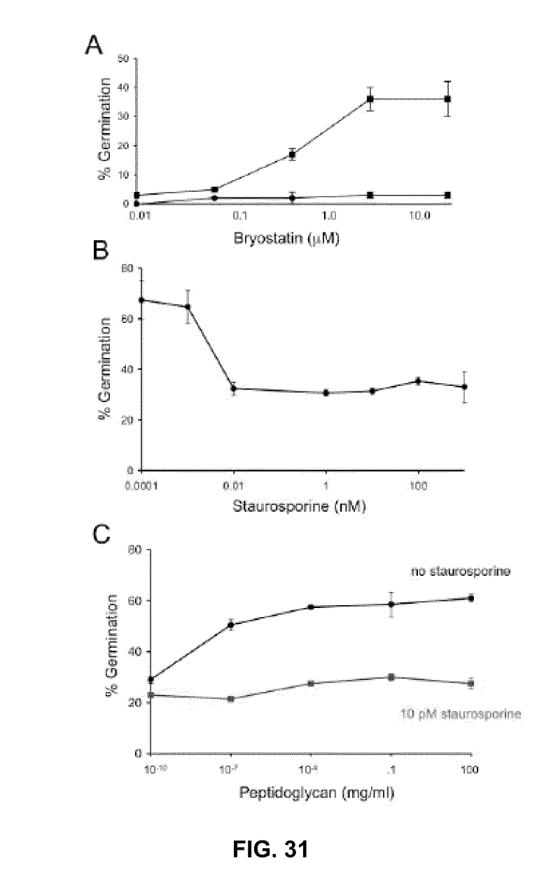

These spores underwent germination, achieving a maximum of ˜40% germination in the presence of 1.0 μM bryostatin (FIG. 31A). Bryostatin treatment of ΔprkC spores had no effect (FIG. 31A), indicating that bryostatin was acting directly on PrkC_{Bs}.

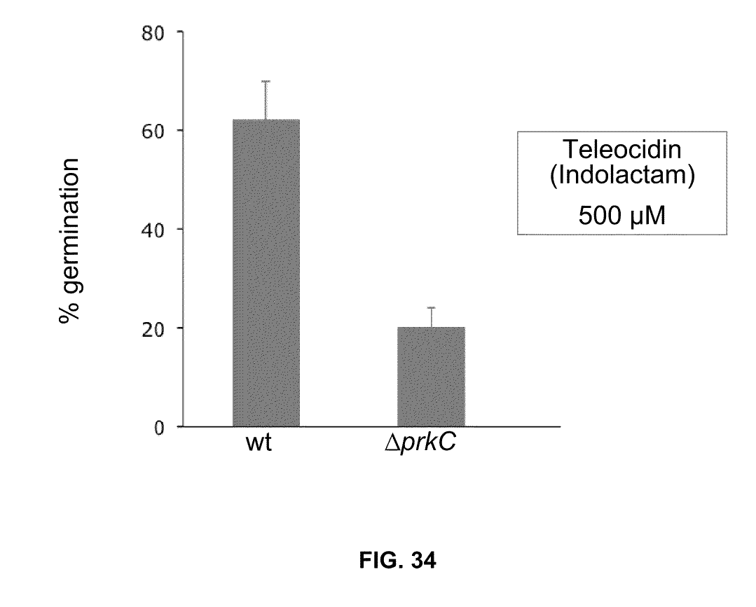

In addition, the molecule (teleocidin) produced by a Streptomyces is a broad-spectrum eukaryotic Ser/Thr activator. Teleocidin was incubated with both wildtype and ΔprkC B. subtilis spores and was able to stimulate germination of only the wildtype spore (see e.g., FIG. 34).

Thus, activation of PrkC is sufficient to induce germination, even in the absence of a germinant.

Dormant spores are resistant to treatments that kill vegetative cells such as antibiotics. However, bryostatin-treated wild type B. subtilis spores become sensitive to the ribosomal antibiotics tetracycline and spectinomycin (Table 2 in Example 6; data not shown). Since these antibiotics are, like bryostatin, small enough to penetrate the spore coat and membrane, dormant spores are probably resistant because they lack the metabolic activity that is the target of these molecules. Thus, bryostatin stimulation of PrkC_{Bs} appears to lead to the resumption of metabolic activity, a hallmark of germination.

Staurosporine, a small molecule ATP mimic, inhibits intracellular eukaryotic Ser/Thr kinases (Ruegg and Burgess, 1989). Similar to the bryostatin experiments, it was asked whether staurosporine would affect PrkC function. Incubation of staurosporine at concentrations as low as 10 pM with spores significantly reduced peptidoglycan-dependent germination (FIG. 31B). In contrast, L-alanine germination was unaffected by staurosporine, consistent with the ability of ΔprkC spores to respond to nutrient germinants (data not shown). Increasing amounts of peptidoglycan did not increase germination in the presence of 10 pM staurosporine, indicating that the compound was not competing for binding of the peptidoglycan (FIG. 31C). Thus, PrkC_{Bs} phosphorylation of a downstream target is essential for transduction of the peptidoglycan germination signal.

### Example 17

### Discussion of Examples 7-14

Metazoans recognize bacterial cells by the presence of microbial-specific molecules such as peptidoglycan that bind to receptors and trigger the activation of cellular pathways mediating the host response to infection (Kaparakis et al., 2007). In addition, peptidoglycan fragments induce cytopathogical changes in the host during bacterial infections and mediate symbiotic interactions between the eukaryotic host and bacteria (Cloud-Hansen et al., 2006). The presence of these molecules is also consistent with the ability of the environment to support microbial growth since they are released by growing bacteria in large quantities. Here, it is reported that supernatants of growing bacteria, peptidoglycan isolated from a wide variety of bacteria, and purified muropeptides induce germination in dormant bacterial spores. Thus, peptidoglycan fragments serve as a novel mechanism of inter-species bacterial signaling that likely indicates the presence of growing bacteria (Bassler and Losick, 2006).

PrkC is necessary for germination in response to muropeptides and it is capable of binding peptidoglycan. The ability of peptidoglycan derived from different bacteria to bind to eukaryotic peptidoglycan recognition proteins (PGRP) is dependent on the identity of a single residue (L-Lys vs. m-Dpm) in the stem peptide (Swaminathan et al., 2006). A similar specificity was observed here in the ability of peptidoglycan to stimulate germination of B. subtilis spores. The structure of a PGRP bound to its peptidoglycan substrate (Chang et al., 2006; Lim et al., 2006) identifies the molecular basis of this specificity. While there is no analogous structure of PrkC_{Bs} bound to peptidoglycan, it is intriguing that the observed substrate specificities of PGRP and PrkC_{Bs} are so similar despite their apparent phylogenetic distance and lack of primary sequence homology. In addition, subunits of PG bind to and activate the Cyr1p adenyl cyclase of Candida albicans, a key component of the hyphal development pathway, suggesting that PG can play a role in non-immunological physiological responses of eukaryotic cells (Xu et al., 2008).

Mechanism of Spore Germination. The ability of purified muropeptides and cell-free supernatant isolated from a variety of bacteria to stimulate germination of B. subtilis spores (FIG. 18A, B) suggests that muropeptides released by growing bacteria are a general signal for germination. Since spores undergo a small but detectable rate of spontaneous germination (Paidhungat and Setlow, 2000), the ability of these germinated spores to grow will be detected by the still dormant spores in the population because of their release of muropeptides. Finally, the inter-species nature of this signal (Table 3) is consistent with the existence of most bacteria in multi-species consortia and suggests that spore-forming bacteria monitor the growth of diverse microbes in their environment.

Spore germination initially involves a series of biophysical and biochemical events including ion fluxes and spore rehydration that quickly lead to a loss of spore heat resistance (Setlow, 2003). PrkC is required for the loss of heat resistance in peptidoglycan-dependent germination where it phosphorylates EF-G, an essential ribosomal GTPase involved in mRNA and tRNA translocation (Savelsbergh et al., 2003). While the effect of phosphorylation on EF-G activity is not known, the activity of eEF-2, the eukaryotic homolog of EF-G, is determined by its phosphorylation state (Ryazanov et al., 1988). Thus, binding of peptidoglycan fragments to the extracellular PASTA-containing domain of PrkC_{Bs} could stimulate translation by inducing the intracellular kinase domain of PrkC to phosphorylate EF-G.

Dormant spores contain mRNA and polysomes (Setlow and Kornberg, 1970) and, when disrupted, they incorporate radiolabeled amino acids (Chambon et al., 1968). Recent evidence indicates that spores contain specific mRNA species directly relevant to the physiological context of the organism (Bettegowda et al., 2006). Thus, translation could be the initial biosynthetic step in the transformation of the dormant spore to a metabolically active cell. However, given the complete metabolic dormancy of the spore core, PrkC_{Bs} phosphorylation of EF-G is unlikely to be the sole cause of germination. PrkC_{Bs} itself, or an unidentified target of the kinase, probably plays a role in the spore rehydration necessary for translation and metabolism.

Chemical Modulation of the Germination Process. The spore-forming bacterium Clostridium difficile causes an increasingly prevalent gastrointestinal colitis that occurs following antibiotic therapy. C. difficile likely survives exposure to antibiotics as spores, since the vegetative form is sensitive to antibiotics (Hecht et al., 2007). When germinated, these spores enter vegetative growth where they are capable of producing the toxins that cause colitis. Interestingly, members of the GerA germination receptor family are absent from the C. difficile genome. However, since there is a PrkC homolog (Sebaihia et al., 2006), this protein may play an essential role in C. difficile germination.

Most clinically relevant antibiotics are derived from soil-dwelling organisms, presumably reflecting inter-bacterial competition within soil. While these compounds typically target essential pathways in growing cells, it was observed that staurosporine acts by blocking germination of dormant spores at very low (˜pM) concentrations. Since staurosporine is synthesized by a species of the soil bacterium Streptomyces (Onaka et al., 2002), it is appealing to posit that staurosporine inhibition of spore germination is relevant to interactions between Streptomyces spp. and Bacillus spp. in the environment.

A conserved pathway for relief of bacterial dormancy. Many bacteria exist in a state of metabolic dormancy (Keep et al., 2006) which increases their resistance to antibiotics or to other stresses found in nutrient limited environments. However, the advantages afforded by this state of dormancy are dependent on the ability of the cell to exit this state when conditions conducive to growth become present. Dormant cells of Micrococcus luteus are stimulated to divide (resuscitate) by exposure to non-dormant M. luteus cells and this stimulation requires the resuscitation-promoting factor (Rpf), a secreted 17-kDa protein that digests peptidoglycan (Mukamolova et al., 2006) into soluble fragments, likely including muropeptides. The ability of the human pathogen M. tuberculosis to reactivate following in vivo latency is affected by the presence of endogenous resuscitation-promoting factors (Tufariello et al., 2006). Since M. tuberculosis PknB is a homolog of PrkC, PknB may also recognize peptidoglycan fragments as a signal that growth-promoting conditions exist and this ability may have important implications for pathogenesis of this organism. Finally, these observations may provide a mechanistic basis for the observation that many microbes require other bacteria in the local environment in order to grow (Kaeberlein et al., 2002).

### Example 18

### PrpC Phosphatase Counters the Effect of PrkC in Spore Germination

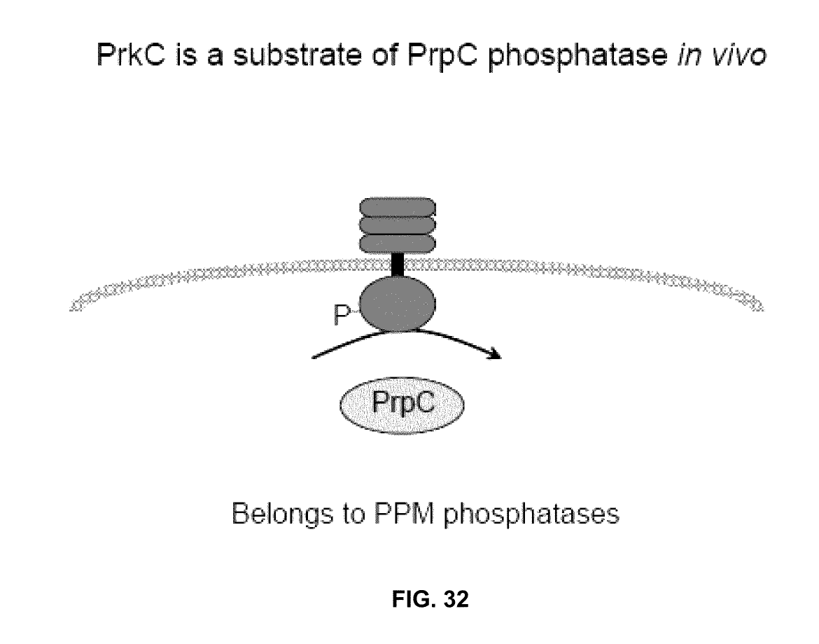

PrpC phosphatase is a PPM-like phosphatase, which are characterized by up to 11 motifs conserved in sequence and spacing (Obuchowski et al., 2000). A substrate of PrpC phosphatase is PrkC (FIG. 32). PrpC and PrkC have opposing physiological roles in stationary phase survival (Gaidenko et al., 2002). It was determined whether the two enzymes also had opposing roles in inducing sporulation.

Methods are according to Example 7 unless indicated otherwise.

For these studies, the following strains were utilized—a ΔprpC mutant, a hyper-expressing PrpC strain, and two mutants of the hyperexpressing PrpC strain that no longer have PrpC activity. These mutants are D36N and D195N.

Results of these germination studies are summarized in FIG. 33. In those studies, the ΔprpC mutant did not affect the ability of the spores to germinate when stimulated by peptidoglycan. However, the strain hyperexpressing PrpC did not germinate under the same conditions. This strain thus behaves as a ΔprkC. Confirming these findings, the D36N and D195N PrpC mutants did not affect germination, thus behaving as the ΔprpC mutant. These results further confirm that the PrpC phosphatase counters the effects of PrkC on sporulation, and that stimulation of PrpC phosphatase can counter the effects of PrkC on germination, apparently by dephosphorylating PrkC. See also Example 5.

### Example 19

### Regulation of Germination by Small Molecule Kinase Modulators

The cytoplasmic domain of the PrkC_{Bs} homolog, M. tuberculosis PknB, is structurally homologous to the catalytic domains of eukaryotic Ser/Thr kinases (Young et al., 2003). This similarity suggests that small molecules known to modulate the activity of these eukaryotic kinases might also modulate PrkC homologs. One of these molecules, bryostatin, a natural product synthesized by a marine bacterium, potently activates eukaryotic intracellular Ser/Thr kinases through direct binding to the phorbol ester binding site (Hale et al., 2002).

It was examined whether bryostatin activated PrkC by incubating wild type B. subtilis spores with a range of bryostatin concentrations.

Methods are according to Example 7 unless indicated otherwise.

These spores underwent germination, achieving a maximum of ˜40% germination in the presence of 1.0 μM bryostatin (see e.g., FIG. 31A). Bryostatin treatment of ΔprkC spores had no effect (FIG. 31A), indicating that bryostatin was acting directly on PrkC_{Bs}. In addition, the molecule (teleocidin) produced by a Streptomyces is a broad-spectrum eukaryotic Ser/Thr activator. Teleocidin was incubated with both wildtype and ΔprkC B. subtilis spores and was able to stimulate germination of only the wildtype spore. Thus, activation of PrkC is sufficient to induce germination, even in the absence of a germinant.

### REFERENCES

Atrih, A., Bacher, G., Allmaier, G., Williamson, M. P., and Foster, S. J. (1999). Analysis of peptidoglycan structure from vegetative cells of Bacillus subtilis 168 and role of PBP 5 in peptidoglycan maturation. J Bacteriol 181, 3956-3966.
Ausubel et al. (2002). Short Protocols in Molecular Biology, 5th ed., Current Protocols, ISBN-10: 0471250929.
Baneyx (2004). Protein Expression Technologies, Taylor & Francis, ISBN-10: 0954523253). Bassler, B. L., and Losick, R. (2006). Bacterially speaking. Cell 125, 237-246.
Bettegowda, C., Huang, X., Lin, J., Cheong, I., Kohli, M., Szabo, S. A., Zhang, X., Diaz, L. A., Jr., Velculescu, V. E., Parmigiani, G., et al. (2006). The genome and transcriptomes of the anti-tumor agent Clostridium novyi-NT. Nat Biotechnol 24, 1573-1580.
Beveridge, T. J. (1999). Structures of Gram-negative cell walls and their derived membrane vesicles. J Bacteriol 181, 4725-4733.
Boneca, I. G. (2005). The role of peptidoglycan in pathogenesis. Curr Opin Microbiol, 8, 46-53. Chambon, P., Deutscher, M. P., and Kornberg, A. (1968). Biochemical studies of bacterial sporulation and germination. X. Ribosomes and nucleic acids of vegetative cells and spores of Bacillus megaterium. J Biol Chem 243, 5110-5116.
Chang, C. I., Chelliah, Y., Borek, D., Mengin-Lecreulx, D., and Deisenhofer, J. (2006). Structure of tracheal cytotoxin in complex with a heterodimeric pattern-recognition receptor. Science 311, 1761-1764.
Cloud-Hansen, K. A., Peterson, S. B., Stabb, E. V., Goldman, W. E., McFall-Ngai, M. J., and Handelsman, J. (2006). Breaching the great wall: peptidoglycan and microbial interactions. Nat Rev Microbiol 4, 710-716.
Cohen-Gonsaud, M., et al. (2004). Resuscitation-promoting factors possess a lysozyme-like domain. Trends Biochem Sci 29, 7-10.
Cohen-Gonsaud, M., Barthe, P., Bargenris, C., Henderson, B., Ward, J., Roumestand, C. and Keep, N. H. (2005) The structure of a resuscitation-promoting factor domain from Mycobacterium tuberculosis shows homology to lysozyes. Nat. Struct. Mol. Biol. 12, 270-3.
D'Elia, M. A., Millar, K. E., Beveridge, T. J., and Brown, E. D. (2006). Wall teichoic acid polymers are dispensable for cell viability in Bacillus subtilis. J Bacteriol 188, 8313-8316.
Dasgupta, A., Datta, P., Kundu, M., and Basu, J. (2006). The serine/threonine kinase PknB of Mycobacterium tuberculosis phosphorylates PBPA, a penicillin-binding protein required for cell division. Microbiology 152, 493-504.
Doyle, R. J., Chaloupka, J., and Vinter, V. (1988). Turnover of cell walls in microorganisms. Microbiol. Rev 52, 554-567.
Driks, A. (1999). Bacillus subtilis spore coat. Microbiol. Mol Biol Rev 63, 1-20.
Eckert, C., Lecerf, M., Dubost, L., Arthur, M., and Mesnage, S. (2006). Functional analysis of AtlA, the major N-acetylglucosaminidase of Enterococcus faecalis. J Bacteriol 188, 8513-8519.
Fernandez, P. et al. (2006). The Ser/Thr protein kinase PknB is essential for sustaining mycobacterial growth. J. Bacteriol. 188, 7778-7784.
Filipe, S. R., Tomasz, A. and Ligoxygakis, P. (2005). Requirements of peptidoglycan structure that allow detection by the Drosophila Toll pathway. EMBO reports 6, 327-333.
Gaidenko, T. A., Kim, T. J., and Price, C. W. (2002). The PrpC serine-threonine phosphatase and PrkC kinase have opposing physiological roles in stationary-phase Bacillus subtilis cells. J Bacteriol 184, 6109-6114.
Gellissen, ed. (2005) Production of Recombinant Proteins: Novel Microbial and Eukaryotic Expression Systems, Wiley-VCH, ISBN-10: 3527310363;
Gescher, A. (1998). Analogs of staurosporine: potential anticancer drugs? General pharmacology 31, 721-728.
Gilmore, M. E., Bandyopadhyay, D., Dean, A. M., Linnstaedt, S. D., and Popham, D. L. (2004). Production of muramic delta-lactam in Bacillus subtilis spore peptidoglycan. J Bacteriol 186, 80-89.
Girardin, S. E., Travassos, L. H., Nerve, M., Blanot, D., Boneca, I. G., Philpott, D. J., Sansonetti, P. J., and Mengin-Lecreulx, D. (2003). Peptidoglycan molecular requirements allowing detection by Nod1 and Nod2. J Biol Chem 278, 41702-41708.
Goodell, E. W., and Schwarz, U. (1985). Release of cell wall peptides into culture medium by exponentially growing Escherichia coli. J Bacteriol 162, 391-397.
Hale, K. J., Hummersone, M. G., Manaviazar, S., and Frigerio, M. (2002). The chemistry and biology of the bryostatin antitumour macrolides. Natural product reports 19, 413-453.
Hasegawa, M., Yang, K., Hashimoto, M., Park, J. H., Kim, Y. G., Fujimoto, Y., Nunez, G., Fukase, K., and Inohara, N. (2006). Differential release and distribution of Nod1 and Nod2 immunostimulatory molecules among bacterial species and environments. J Biol Chem 281, 29054-29063.
Hecht, D. W., Galang, M. A., Sambol, S. P., Osmolski, J. R., Johnson, S., and Gerding, D. N. (2007). In vitro activities of 15 antimicrobial agents against 110 toxigenic Clostridium difficile clinical isolates collected from 1983 to 2004. Antimicrob Agents Chemother 51, 2716-2719.
Hudson, K. D., Corfe, B. M., Kemp, E. H., Feavers, I. M., Coote, P. J., and Moir, A. (2001). Localization of GerAA and GerAC proteins in the Bacillus subtilis spore. J Bacteriol 183, 4317-4322.
Hungerer, K. D., and Tipper, D. J. (1969). Cell wall polymers of Bacillus sphaericus 9602.1. Structure of the vegetative cell wall peptidoglycan. Biochemistry 8, 3577-3587.
Jones, G., and Dyson, P. (2006). Evolution of transmembrane protein kinases implicated in coordinating remodeling of Gram-positive peptidoglycan: inside versus outside. J Bacteriol 188, 7470-7476.
Jacobs, C., Frere, J., and Nomard, S. (1997). Cytosolic Intermediates for Cell Wall Biosynthesis and Degradation Control Inducible 13-lactam resistance in Gram-negative bacteria. Cell 88, 823-832.
Kaeberlein, T., Lewis, K., and Epstein, S. S. (2002). Isolating “uncultivable” microorganisms in pure culture in a simulated natural environment. Science 296, 1127-1129.
Kaparakis, M., Philpott, D. J., and Ferrero, R. L. (2007). Mammalian NLR proteins; discriminating foe from friend. Immunology and cell biology 85, 495-502.
Kaprelyants, A. S., and Kell, D. B. (1993). Dormancy in stationary-phase cultures of Microcococcus luteus: flow cytometric analysis of starvation and resuscitation. Appl. Environ. Microbiol. 59, 3187-3196.
Keep, N. H., Ward, J. M., Cohen-Gonsaud, M., and Henderson, B. (2006). Wake up! Peptidoglycan lysis and bacterial non-growth states. Trends Microbiol 14, 271-276.
Koropatnick, T. A., Engle, J. T., Apicella, M. A., Stabb, E. V., Goldman, W. E., and McFall-Ngai, M. J. (2004). Microbial Factor-Mediated Development in a Host-Bacterial Mutualism. Science 306, 1186-1188.
Lim, J. H., Kim, M. S., Kim, H. E., Yano, T., Oshima, Y., Aggarwal, K., Goldman, W. E., Silverman, N., Kurata, S., and Oh, B. H. (2006). Structural basis for preferential recognition of diaminopimelic acid-type peptidoglycan by a subset of peptidoglycan recognition proteins. J Biol Chem 281, 8286-8295.
Madec, E., Laszkiewicz, A., Iwanicki, A., Obuchowski, M., and Seror, S. (2002). Characterization of a membrane-linked Ser/Thr protein kinase in Bacillus subtilis, implicated in developmental processes. Mol Microbiol 46, 571-586.
Mauck, J., Chan, L., and Glaser, L. (1971). Turnover of the cell wall of Gram-positive bacteria. J Biol Chem 246, 1820-1827.
McPherson, D. C., and Popham, D. L. (2003). Peptidoglycan synthesis in the absence of class A penicillin-binding proteins in Bacillus subtilis. J Bacteriol 185, 1423-1431.
Mukamolova, G. V., et al. (2002). The rpf gene of Micrococcus luteus encodes an essential secreted growth factor. Mol Microbiol 46, 611-21.
Mukamolova, G. V., Murzin, A. G., Salina, E. G., Demina, G. R., Kell, D. B., Kaprelyants, A. S., and Young, M. (2006). Muralytic activity of Micrococcus luteus Rpf and its relationship to physiological activity in promoting bacterial growth and resuscitation. Mol Microbiol 59, 84-98.
Nicholson, W. L., Munakata, N., Horneck, G., Melosh, H. J., and Setlow, P. (2000). Resistance of Bacillus endospores to extreme terrestrial and extraterrestrial environments. Microbiol. Mol Biol Rev 64, 548-572.
Obuchowski, M. et al. (2000). Characterization of PrpC from Bacillus subtilis, a member of the PPM phosphatase family. J Bacteriol 182, 5634-5638.
Onaka, H., Taniguchi, S., Igarashi, Y., and Furumai, T. (2002). Cloning of the staurosporine biosynthetic gene cluster from Streptomyces sp. TP-A0274 and its heterologous expression in Streptomyces lividans. J Antibiotics 55, 1063-1071.
Paidhungat, M., and Setlow, P. (2000). Role of ger proteins in nutrient and nonnutrient triggering of spore germination in Bacillus subtilis. J Bacteriol 182, 2513-2519.
Paidhungat, M., and Setlow, P. (2001). Localization of a germinant receptor protein (GerBA) to the inner membrane of Bacillus subtilis spores. J Bacteriol 183, 3982-3990.
Park, J. T. (1995). Why does Escherichia coli recycle its cell wall peptides? Mol. Microbiol. 17, 421-6.
Ragavani, A., Finan, C. L., and Young, M. (2005). A novel firmicute protein family related to the actinobacterial resuscitation-promoting actors by nonorthologous domain. BMC Genomics 6, 39-52.
Ruegg, U. T., and Burgess, G. M. (1989). Staurosporine, K-252 and UCN-01: potent but nonspecific inhibitors of protein kinases. Trends in pharmacological sciences 10, 218-220.
Ryazanov, A. G., Shestakova, E. A., and Natapov, P. G. (1988). Phosphorylation of elongation factor 2 by EF-2 kinase affects rate of translation. Nature 334, 170-173.
Santelli, E., Liddington, R. C., Mohan, M. A., Hoch, J. A., and Szurmant, H. (2007). The crystal structure of Bacillus subtilis Yycl reveals a common fold for two members of an unusual class of sensor histidine kinase regulatory proteins. J Bacteriol 189, 3290-3295.
Sambrook and Russel (2006). Condensed Protocols from Molecular Cloning: A Laboratory Manual, Cold Spring Harbor Laboratory Press, ISBN-10: 0879697717
Sambrook and Russel (2001) Molecular Cloning: A Laboratory Manual, 3d ed., Cold Spring Harbor Laboratory Press, ISBN-10: 0879695773
Savelsbergh, A., Katunin, V. I., Mohr, D., Peske, F., Rodnina, M. V., and Wintermeyer, W. (2003). An elongation factor G-induced ribosome rearrangement precedes tRNA-mRNA translocation. Molecular cell 11, 1517-1523.
Schleifer, K. H., and Kandler, 0. (1972). Peptidoglycan types of bacterial cell walls and their taxonomic implications. Bacteriological reviews 36, 407-477.
Setlow, P. (2003). Spore germination. Curr Opin Microbiol 6, 550-556.
Setlow, P., and Kornberg, A. (1970). Biochemical studies of bacterial sporulation and germination. 23. Nucleotide metabolism during spore germination. J Biol Chem 245, 3645-3652.
Sharma, K., Gupta, M., Krupa, A., Srinivasan, N., and Singh, Y. (2006). EmbR, a regulatory protein with ATPase activity, is a substrate of multiple serine/threonine kinases and phosphatase in Mycobacterium tuberculosis. The FEBS journal 273, 2711-2721.
Shatalin, K. Y., and Neyfakh, A. A. (2005). Efficient gene inactivation in Bacillus anthracis. FEMS Microbiol Lett 245, 315-319.
Steen, A., Buist, G., Leenhouts, K. J., El Khattabi, M., Grijpstra, F., Zomer, A. L., Venema, G., Kuipers, O. P., and Kok, J. (2003). Cell wall attachment of a widely distributed peptidoglycan binding domain is hindered by cell wall constituents. J Biol Chem 278, 23874-23881.
Studier (2005) Protein Expr Purif. 41, 207-234.
Swaminathan, C. P., Brown, P. H., Roychowdhury, A., Wang, Q., Guan, R., Silverman, N., Goldman, W. E., Boons, G. J., and Mariuzza, R. A. (2006). Dual strategies for peptidoglycan discrimination by peptidoglycan recognition proteins (PGRPs). Proc Natl Acad Sci USA 103, 684-689.
Tufariello, J. M., Mi, K., Xu, J., Manabe, Y. C., Kesavan, A. K., Drumm, J., Tanaka, K., Jacobs, W. R., Jr., and Chan, J. (2006). Deletion of the Mycobacterium tuberculosis resuscitation-promoting factor Rv1009 gene results in delayed reactivation from chronic tuberculosis. Infect Immun 74, 2985-2995.
Yeats, C., Finn, R. D., and Bateman, A. (2002). The PASTA domain: a beta-lactam-binding domain. Trends Biochem Sci 27, 438.
Votyakova, T. V., A. S. Kaprelyants, and D. B. Kell (1994). Influence of viable cells on the resuscitation of dormant cells in Micrococcus luteus cultures held in an extended stationary phase: the Population Effect. Appl Environ Microbiol 60, p. 3284-3291.
Young, T. A., Delagoutte, B., Endrizzi, J. A., Falick, A. M., and Alber, T. (2003). Structure of Mycobacterium tuberculosis PknB supports a universal activation mechanism for Ser/Thr protein kinases. Nature structural biology 10, 168-174.

What is claimed is:

1. A method of treating an infection of a first spore-forming Gram-positive bacterium comprising:
administering to a subject in need thereof an effective amount of
(A) a first composition comprising at least one of
(i) a peptidoglycan fragment or muropeptide of a second bacterium or a preparation comprising cell walls from the second bacterium;
(ii) a compound that stimulates activity of a bacterial serine/threonine protein kinase; or
(iii) a compound that inhibits activity of a bacterial PPM-like phosphatase;
wherein said first composition stimulates germination of the spore; or
(B) a second composition comprising at least one of
(iv) a compound that inhibits activity of a bacterial serine/threonine protein kinase; or
(v) a compound that stimulates activity of a PPM-like phosphatase of a first Gram-positive bacterium;
wherein said second composition inhibits germination of the spore.

2. The method of claim 1 further comprising administering to the subject an antibiotic effective against the first Gram-positive bacterium.

3. The method of claim 1, wherein the first Gram-positive bacterium is selected from the group consisting of Bacillus, Clostridium, Desulfotomaculum, Sporolactobacillus, Sporosarcina, and Thermoactinomyces.

4. The method of claim 3, wherein the first Gram-positive bacterium is selected from the group consisting of B. anthracis, B. cereus, B. thuringiensis, C. difficile, C. botulinum, B. subtilis, B. megaterium, B. anthracis, and C. acetobutylicum.

5. The method of claim 1, wherein the second bacterium is a second Gram-positive bacterium.

6. The method of claim 5, wherein the second Gram-positive bacterium is selected from the group consisting of Bacillus, Clostridium, Listeria, and Streptomyces.

7. The method of claim 6, wherein the second Gram-positive bacterium is selected from the group consisting of B. subtilis, B. megaterium, B. anthracis, C. acetobutylicum, L. monocytogenes, and S. coelicolor.

8. The method of claim 5, wherein the first Gram-positive bacterium is of the same genus or the same species as the second Gram-positive bacterium.

9. The method of claim 1, wherein the second bacterium is a second Gram-negative bacterium.

10. The method of claim 9, wherein the second Gram-negative bacterium is E. coli.

11. The method of claim 1, wherein the preparation of cell walls comprises purified peptidoglycan fragments or purified muropeptides.

12. The method of claim 11, wherein the purified peptidoglycan fragments or purified muropeptides comprise a diaminopimelic acid (DAP) in the third residue of the stem-peptide.

13. The method of claim 1, wherein the preparation of cell walls does not contain a living bacterium.

14. The method of claim 1, wherein the compound that stimulates activity of a bacterial serine/threonine protein kinase is a compound that stimulates activity of PrkC or a polypeptide having an amino acid sequence at least 25% identical to the sequence encoded by a complement of nucleotides 2106-4033 of SEQ ID NO:1.

15. The method of claim 1, wherein the compound that stimulates activity of a bacterial serine/threonine protein kinase is selected from the group consisting of a phorbol ester; phorbol-12-myristate-13-acetate (PMA); a bryostatin; and teleocidin; or a derivative thereof.

16. The method of claim 2, wherein the antibiotic is selected from the group consisting of a beta-lactam, clavulanic acid, a monobactam, a carboxypenem, an aminoglycoside, gentamicin, a glycopeptide, a lincomycin, a macrolide, bacitracin, rifamycin, tetracycline, chloramphenicol, penicillin G, benzathine benzylpenicillin, phenoxymethylpenicillin, amoxicillin, ampicillin, bacampicillin, pivampicillin, clavulanic acid, aztreonam, imipenem, streptomycin, vancomycin, clindamycin, erythromycin, bacitracin, rifampicin, doxycycline, tigecycline, chloramphenicol, linezolid, quinupristin-dalfopristin, and daptomycin.

17. The method of claim 2, wherein a composition comprising the antibiotic and at least one of (A)(i), (A)(ii), or (A)(iii) is administered to the subject.

18. The method of claim 1, wherein the subject is a mammal.

19. The method of claim 1, wherein the subject is selected from the group consisting of a mouse, rat, guinea pig, gerbil, dog, cat, sheep; cow; horse; pig; goat; donkey; mule; monkey; prosimian; ape; and human.

20. The method of claim 1, wherein the (B)(iv) compound that inhibits activity of a bacterial serine/threonine protein kinase is selected from the group consisting of adaphostin, AG 490, AG 825, AG 957, AG 1024, aloisine, aloisine A, alsterpaullone, aminogenistein, API-2, apigenin, arctigenin, AY-22989, bisindolylmaleimide IX, chelerythrine, DMPQ, DRB, edelfosine, erbstatin analog, ET18OCH3, ERK inhibitor fasudil, gefitinib, H-7, H-8, H-89, HA-100, HA-1004, HA-1077, HA-1100, Heatstable protein kinase A inhibitor PKI, hydroxyfasudil, indirubin-3′-oxime, 5-lodotubercidin, kenpaullone, KN-62, KY12420, LFM-A13, luteolin, LY-294002, LY294002, mallotoxin, ML-9, NSC-154020, NSC-226080, NSC-231634, NSC-664704, NSC-680410, NU6102, olomoucine, oxindole I, PD 153035, PD 98059, PD 169316, phloridzin, piceatannol, picropodophyllin, PKI, PP1, PP2, purvalanol A, quercetin, RAPA, rapamune, rapamycin, Ro 31-8220, roscovitine, rottlerin, SB202190, SB203580, sirolimus, SL327, SP600125, staurosporine, STI-571, SU1498, SU4312, SU6656, syk inhibitor, TBB, TCN, triciribine, Tyrphostin AG 490, Tyrphostin AG 825, Tyrphostin AG 957, Tyrphostin AG 1024, U0126, W-7, wortmannin, Y-27632, ZD 1839, and ZM 252868.

21. A pharmaceutical composition comprising:
an antibiotic effective against a Gram-positive bacterium; and
at least one of
(A) a first composition comprising at least one of
(i) a peptidoglycan fragment or muropeptide of a second bacterium or a preparation comprising cell walls from the second bacterium;
(ii) a compound that stimulates activity of a serine/threonine protein kinase of a Gram-positive bacterium; or
(iii) a compound that inhibits activity of a PPM-like phosphatase of a Gram-positive bacterium;
wherein said first composition stimulates germination of a Gram-positive bacterium spore; or
(B) a second composition comprising at least one
(iv) a compound that inhibits activity of a bacterial serine/threonine protein kinase; or
(v) a compound that stimulates activity of a PPM-like phosphatase of a first Gram-positive bacterium;
wherein said second composition inhibits germination of a Gram-positive bacterium spore; and
a pharmaceutically acceptable carrier or excipient.

22. A method of decontaminating an environment containing spores of a first Gram-positive bacterium comprising:
treating the environment with a composition comprising at least one of
(i) a peptidoglycan fragment or muropeptide of a second bacterium or a preparation comprising cell walls from the second bacterium;
(ii) a compound that stimulates activity of a serine/threonine protein kinase of a Gram-positive bacterium; or
(iii) a compound that inhibits activity of a PPM-like phosphatase of a Gram-positive bacterium.

23. A method of modulating germination of a spore of a first Gram-positive bacterium comprising:
contacting the spore of the first Gram-positive bacterium with
(A) a first composition comprising at least one of
(i) a peptidoglycan fragment or muropeptide of a second bacterium or a preparation comprising cell walls from the second bacterium;
(ii) a compound that stimulates activity of a serine/threonine protein kinase of a Gram-positive bacterium; or
(iii) a compound that inhibits activity of a PPM-like phosphatase of a Gram-positive bacterium;
wherein said first composition stimulates germination of the spore; or
(B) a second composition comprising at least one
(iv) a compound that inhibits activity of a bacterial serine/threonine protein kinase; or
(v) a compound that stimulates activity of a PPM-like phosphatase of a first Gram-positive bacterium;
wherein said second composition inhibits germination of the spore.


SEQUENCE LISTING (8 sequences)

SEQ ID NO: 1 - DNA, 9768 bp, Artificial
  Plasmid
aacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgaaatagtacataatggatttccttacgcgaaatacgggcagacatggcctgcccggttattattatttttgacaccagaccaactggtaatggtagcgaccggcgctcaggatcctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccgaattagcttgcatgcctactggccgaacctcagcgtgatgatgccgtcccggttgacgccggtccccgccggcgggttttgatagacgacccggttgtgttgggagccaccggcgtcgacgtcggcccctttgtcgagcatcccggtccagcccagcgcgcgcaatcgtggttcggcgtcgacccagaacatgccggataggtcgggcatgacgaattggttgcccttggacacctgtagttcgatgactgaatcgaccggaactgtggtgcctgcgggtggattggtgccggtcacctcgccggcgggacgggggctgtccaccgaggcctgactgaatttggtgaagccgtagacgttgaggttcttctgcgccacgtcgacggtctggcccgcgacatcgggaatgtctttggtcgccggaccagagccaacgatgatgatgaccacattggtgatggccgacgtctggttggctggcgggttggtcccgatgaccttgcccaccagttccggggtggacggcgaattcgcttgcttgaagcggccgaatccggcggcagtcagtttcttgaccgcttcggcgtatgtcagcgtggagacgtcgggtatttcgcgttgctcgggtccggtggacacgttgactgtgatctcgtcgcctgcactcaccgacgtgttggcggccgggtcggtgccgataacgtggtccggtgggattgtcgagtccggcttctgcaaggtgcggattttgaagccccggttttgcagtgtggcgatggcgtcggcggaggattgaccccgaacgtcgggaacttgaacgtcgcgggtgatgccgccgaacgtgttgatggcgatggttaccacgacggtcagcacagcgagcacggcgaccaccgcaacccaacggcccaccgaaccgatgctgcggtcacggtcggtgtcgtctaagtcctggcgtggtagcggatcggtgcgcggaccgctaaggttgccggccgcagacgacagcagcgaggtccgctcggcatcggtgagcactttgggcgcctcgggcggctcaccgttgtgcacgcggaccaggtcggcgcgcatctccgccgctgtctgatagcggttttccggatttttggccagcgccttgagaacgacggcgtccaggtcggcggagaggccttcgtgccgcgccgaaggtgggatcgggtcttcgcgcacatgttggtaggcaaccgagacgggtgagtcgccggtgaaaggtggctccccggtgaggacttcataaagaacacagcccaaggaatagacatcggatcgggcgtcgacggaatcaccccgggcctgttcgggtgacaggtactgcgccgtgccgatcactgctgcggtctgggtcacgctgttgccgctgtcggcaatggcgcgggcgatgccgaaatccatcacctttactgcattggtcgcgctgatcatgatgttcgccggcttgacgtcacggtggatgattccgttctgatgactgaagttcagcgcttggcaggcgtcggcgatgacctcgatggcgcgtttgggcgtcatcggcccttcggtgtggacaatgtcgcgcagggtaacgccgtcgacgtattccatgacgatgtagggcaatggcccggcgggcgtttcggcttcaccggtgtcgtagaccgcgacgattgcagggtggttcaatgccgcggcgttttgcgcctcacgccggaagcgaaggtaaaaactgggatcgcgggctagatcagcgcgcagcaccttgaccgcaacgtcgcggtgcaaccggaggtcgcgggccaggtggacctcggacatgcccccaaatccaaggatttcgccaagttcgtagcggtcggacaggtgggaaggggtggttttgtcgtcgtcgtctttatagtccattgatcttcaccctcttcaacttggctagctgtcgactaagcttaattgttatccgctcacaattacacacattatgccacaccttgtagataaagtcaacaacttttgcaaaatgaattgtgagtgctcacatttaccctcgagcaacgttcttgccattgctgcataaaaaacgcccggcggcaaccgagcgttctgaattaattaatcatcgggaagatcttcatcaccgaaacgcggcaggcagctctagagttaacaagagtttgtagaaacgcaaaaaggccatccgtcaggatggccttctgcttagctagagcggcggatttgtcctactcaggagagcgttcaccgacaaacaacagataaaacgaaaggcccagtctttcgactgagcctttcgttttatttgatgcctcaagctagagagtcgaattcctgcagccctggcgaatggcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatagggggatcatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagacgacaggcgtttaaggatggagatcaattcacaatcggaaaaggagatccaatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctactgaagtggatttctttaagagctcctttaacttcctcaccagtagttgtatcggtaccataagtagaagcagcaacccaagtagctttaccagcatccggttcaaccagcatagtaagaatcttactggacatcggcagttcttcgaacagtgcgccaactaccagctctttctgcagttcattcagggcaccggagaacctgcgtgcaatccatcttgttcaatcatgcgaaacgatcctcatcctgtctcttgatccatggattacgcgttaacccgggcccgcggatgcatatgatcagatcttaaggcctaggtctagagtctttgttttgacgccattagcgtacgtaacaatcctcgttaaaggacaaggacctgagcggaagtgtatcgtacagtagacggagtatactagtatagtctatagtccgtggaattattatatttatctccgacgatattctcatcagtgaaatccagctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 2 - protein, 59 aa, Escherichia coli
GRASVASDRSIIPPGTTLLAEVPLLDNNFNGQYELRLMVALDVGGAIKGQHFDIYQGIG

SEQ ID NO: 3 - protein, 47 aa, Bacillus subtilis
NAKVIAVDPNVIPLGSKVYVEGYGEATAADTGGAIKGNKIDVFVPSK

SEQ ID NO: 4 - protein, 46 aa, Bacillus anthracis
YSTIAADLRVFPIGTILFVPGYGYGVVADKGGAIKGNRLDLYYDTV

SEQ ID NO: 5 - protein, 47 aa, Listeria monocytogenes
GMKVIAVDPNVIPLGSKVWVEGYGEAIAGDTGGVIKGNIVDVYFPNE

SEQ ID NO: 6 - protein, 47 aa, Clostridium acetobutylicum
GLSTIAVDPRVIPLGTKVYVEGYGYAVAEDTGGAIKNNIIDLFLNSA

SEQ ID NO: 7 - protein, 60 aa, Caulobacter crescentus
PGRAIAVDPGYHAYGGFYWLDAAAPKLVGAFPVYRRAVTALDTGGAIKGEVRADLYMGSG

SEQ ID NO: 8 - protein, 56 aa, Pseudomonas syringe
PGYSVAIDRKVIPLGSLLWLSTTRPDGSSVVRPVAAQDTGGAIAGEVRADLFWGTG